(19) United States
(12) Patent Application Publication
(10) Pub. No.: US 2018/0353108 A1
(43) Pub. Date: Dec. 13, 2018

(54) SYSTEM AND METHOD FOR MONITORING PERSONAL ACTIVITY

(71) Applicant: Alexandre Prate, Frenchs Forest (AU)

(72) Inventor: Alexandre Prate, Frenchs Forest (AU)

(73) Assignee: REALIFEX PTY. LTD., Frenchs Forest (AU)

(21) Appl. No.: 15/838,235

(22) Filed: Dec. 11, 2017

(30) Foreign Application Priority Data
Dec. 9, 2016 (AU) 2016269565

(51) Int. Cl. A61B 5/11; H04L 29/08; H04L 12/58; G06F 9/451

(52) U.S. Cl. CPC A61B 5/1118; H04L 67/22; G06Q 50/01; H04L 67/306; G06F 9/451; H04L 51/02

(57) ABSTRACT

The present invention relates to a system and method for monitoring personal activity and providing feedback on personal well-being.

The system comprises a computing apparatus which is able to receive input activity data relating to an experience of a user. This user activity data may relate to any activity, work-related, exercise related or any other.

The system is also able to obtain contextual data and associate it with the user input activity data. The contextual data may comprise any physical data associated with the user or the environment. For example, it could comprise time, location, heart rate, physical activity of the user and other contextual information.

### RELATED APPLICATION

This application claims priority to Australian patent application no. 2016269565, entitled A SYSTEM AND METHOD FOR MONITORING PERSONAL ACTIVITY, filed on Dec. 9, 2016, which is incorporated herein in its entirety.

### FIELD OF THE INVENTION

The present invention relates to a system and method for monitoring personal activity and, particularly, but not exclusively, to a system and method for monitoring personal activity and providing feedback on personal wellbeing.

### BACKGROUND OF THE INVENTION

Personal “coaches” are well known for providing feedback and advice to clients on the client's personal well-being. These “life coaches” may hold regular meetings with their clients to discuss the client's daily habits, work life, social and other aspects of their life. They may provide feedback and advice to assist their clients in improving aspects of their lives. Such personal life coaches are now quite popular. They are, however quite expensive. Also, they are not always available, so, for example, a client can only meet them and obtain advice occasionally.

It is known for personal coaches to use mobile computing technology to receive information from clients and respond to clients, but technology in this context is used merely in its conventional sense as a communications medium (e.g. email, messaging, etc.).

Some computer based systems are known which enable a user/client to enter personal activity information, such as their experiences in carrying out a particular task or relating to a particular experience. These are very much in the nature of diaries, however, that a user can refer to later on to self-determine what experiences they consider to be positive and useful. Such input could also be provided to a personal coach to obtain their feedback. Again, however, computing technology is used only in a conventional way.

### SUMMARY OF THE INVENTION

In accordance with a first aspect, the present invention provides a system for monitoring personal activity, comprising a computer processor, a memory and an operating system arranged to support computer processes, a user data input process arranged to receive user input activity data, and a contextual data process, arranged to receive contextual data generated by a contextual data generating device, and to associate the contextual data with the user activity data.

In an embodiment, user activity data may comprise data on an activity being performed by user, such as work related activity, exercise related, attendance at a social event or any other activity. It may comprise information input by the user, in the form of a “note” on the activity, for example. In embodiments, it may comprise an emotion experienced by the user (e.g. happy, sad, etc.), how the user feels about the experience (e.g. “impact” of the experience). It may comprise any other information input by the user on the activity.

The contextual data may comprise any physical data associated with the user or the environment, which can be captured by the contextual data generating device. For example, it could comprise time, date, location, heart rate or other medical information that could be captured by a monitor (e.g. blood pressure), weather conditions, temperature, physical activity of the user (e.g. steps, calories burned, the amount of sleep, etc.) It could comprise any other type of physical/environmental information.

The contextual data generating device may be any device which could provide such data. For example it may be a portable computing device, such as a smartphone, laptop, etc. that can obtain location, time, date, weather conditions (using appropriate weather determining applications) and other contextual data. It may comprise an exercise monitor, such as a smartwatch, that can monitor heart rate, blood pressure, steps, calories burned and other physical information. It may comprise any other device.

In an embodiment, the system comprises an analysis and suggestion process, which is arranged to analyse the user input data together with the contextual data and determine suggestions based on the user input data and the contextual data. In an embodiment, the suggestion process is arranged to present suggestions to the user via a user interface.

In an embodiment, the system has the advantage that it effectively provides an automated, sophisticated “life coach” that can record experiences and provide suggestions based on user input. Further, it can utilise computing technology to combine context with the user input (e.g. what's the weather like, time, what is the physical activity of the user, etc.). It can then analyse the context and the user input and automatically provide feedback suggestions to the user based on context and user input experience.

In an embodiment, the user data input process may present various user interfaces to the user to facilitate user input. One interface may comprise a matrix interface which enables the user to move a cursor across the screen and input the “impact” of the event and also their “mood” at the same time.

In an embodiment, the system comprises a device which is arranged to receive the user input. The device may comprise a portable device, such as a smartphone, smart watch, tablet or any other portable computing device. In an embodiment, the device includes an interface for presenting suggestions to the user. The interface may present other material to the user.

In an embodiment, the system comprises a host computing device which is arranged to analyse the user data and contextual data and generate the response, suggestion.

In an embodiment, the user is able to enter contextual data and the system is arranged to retrieve user activity data associated with that contextual data. A user may, therefore, enter contextual time, location, weather or other data and receive back user activity data that is associated with that contextual data.

In an embodiment, the user data input process may enable a user to designate a time in the past. The system is arranged to retrieve contextual data from that time in the past and the user can enter user input activity data for that time in the past and it will be associated with the retrieved contextual data.

In an embodiment, the system further comprises a dimension data process arranged to enable association of dimension data with the user input data. Dimension data may include “categories” such as, in this embodiment, “emotion”, “experience”, “discovery”, “action”, “decision”. In an embodiment, the user input data is stored with the association to the dimension data, and is also stored with the association to contextual data.

In an embodiment, the dimension data process enables the addition of further dimensions. The user interface process may enable the user to add further dimension categories, for example, which they can then use to associate with the user input activity data.

In an embodiment, the user interface process is arranged to generate one or more user interfaces enabling user selection of one or more dimensions to associate their user activity input data with. The user interface may enable the user to input further dimension data. In an embodiment, the analysis and suggestion process may be arranged to analyse the dimension data to determine suggestions to present to the users via the user interface. The suggestion process may analyse the user input activity data, any contextual data and dimension data in order to determine suggestions to present to the user.

In an embodiment, the analysis and suggestion process facilitates an automatic adjustment of the user interface via the user interface process, to present selected interfaces to the user. In an embodiment, the presented interfaces may depend on dimension data and/or context data associated with user activity input data. Depending on the dimension and/or context data, therefore, a particular interface may be presented to the user via the user interface process. This may be done automatically so that a user is provided with information by the suggestion process via the interface.

For example, in one embodiment the suggestion process may determine that the user is entering particular user activity data in a particular geographical location, it may adjust the interface by the user interface process to present a map interface to the user showing the location where the user inputs the particular user activity data.

In an embodiment, the system is arranged to calculate a “Score” which may be associated with a user, which may quantify their interaction with the system. For example, it may quantify their “progress” in life. In an embodiment, different Scores may be calculated depending on user activity data, dimension data and context data. A plurality of different Scores may be calculated for the user, depending on the user activity data, dimension data, and context data. In an embodiment, a Score is automatically presented to the user, by the Analysis and suggestion process, depending on context and/or dimension and user activity data.

In an embodiment, the system comprises a search process, which enables the user, via the user interface process, to search user activity data and other data. In an embodiment, the user interface process may enable the user to search via context and/or dimension data. The user may focus on a particular dimension and/or context, therefore, and retrieve input user activity data associated with that context and/or dimension data

In an embodiment, the suggestion process may comprise software filters and software groupings, and templates enabling suggestion data to be entered, including suggestion text and associated dimension data and/or context data.

In an embodiment, the system comprises an instructor data input process and an instructor user interface process. In an embodiment, these processes operate to enable instructor users to interface with the system 1 and to input instructor data. An instructor may, for example, be “life coach” who wishes to interact with the system and users of the system. This advantageously enables life coaches to utilise the system to monitor data of the user “clients” as client's input the data and to interact with them at any time to provide, for example, life coach advice and suggestions.

In an embodiment, the instructor user interface process is enabled to present interfaces relating to data of clients of the instructor (“being users of the system”).

In an embodiment, where the user input data process enables the user to designate a time in the past, the system may be arranged to retrieve dimension data and/or contextual data from that time in the past and the user can enter input activity data for that time in the past and it will be associated with the retrieved contextual and/or dimension data.

In accordance with a second aspect, the present invention provides a device for monitoring personal activity, comprising a computer processor, a memory and an operating system arranged to support computer processes, a user data input process arranged to receive input data, and a contextual data process, arranged to receive generated contextual data, and a communications arrangement arranged to transmit the contextual data and user activity data to a remote apparatus.

In accordance with a third aspect, the present invention provides a computing apparatus, arranged to receive user input activity data and contextual data generated remotely, and to analyse the user input data together with the contextual data and determine suggestions, and provide the suggestions to a remote device.

In accordance with a fourth aspect, the present invention provides a system for monitoring personal activity, comprising a device in accordance with the second aspect and an apparatus in accordance with the third aspect.

In accordance with a fifth aspect, the present invention provides a method of monitoring personal activity, comprising receiving user input activity data, and receiving generated contextual data, and associating the contextual data with the user activity data.

In an embodiment, the method comprises the further step of analysing the user input activity data and contextual data, and generating suggestions based on the user input data and contextual data.

In accordance with a sixth aspect, the present invention provides a computer program, comprising instructions for implementing a system of the first aspect, a device of the second aspect, or an apparatus of the third aspect of the invention.

In accordance with a seventh aspect, the present invention provides a computer readable medium, providing a computer program in accordance with the second aspect.

In accordance with an eighth aspect, the present invention provides a data signal, comprising a computer program in accordance with the sixth aspect.

In accordance with a ninth aspect, the present invention provides a system for monitoring personal activity, comprising a computer processor, a memory and an operating system arranged to support computer processes, a user data input process arranged to receive user input activity data, a user interface process arranged to generate user interfaces for presentation to a user, and an analysis process and suggestion process, arranged to analyse the user input activity data and control the user interface process to generate user interfaces in dependence on the analysis of the user input activity data.

In accordance with a tenth aspect, the present invention provides a system for monitoring personal activity, comprising a computer processor, a memory and an operating system arranged to support computer processes, a user data input process arranged to receive user input activity data, a user interface process arranged to generate user interfaces for presentation to a user, and an instructor data input process arranged to receive instructor data for control of user interfaces for presentation to the user, whereby instructor data may be presented to a user.

In accordance with an eleventh aspect, the present invention provides a system for monitoring personal activity, comprising a computer processor, a memory and an operating system arranged to support computer processes, a user data input process arranged to receive user input activity data, a dimension data process arranged to generate dimension data and associate the dimension data with the user input activity data, the dimension data comprising a plurality of dimension data categories.

In accordance with a twelfth aspect, the present invention provides a method of monitoring personal activity, comprising the steps of receiving user input activity data, generating user interfaces for presentation to a user, and analysing the user input activity data and controlling the user interface to present user interfaces in dependence on the analysis of the user input activity data.

In accordance with a thirteenth aspect, the present invention provides a method for monitoring personal activity, comprising the steps of receiving user input activity data, generating user interfaces for presentation to a user, and receiving instructor data for control of the user interfaces for presentation to the user.

In accordance with a fourteenth aspect, the present invention provides a method for monitoring personal activity, comprising the steps of receiving user input activity data, generating dimension data and associating the dimension data with the user input activity data, wherein the dimension data comprises a plurality of dimension data categories.

### BRIEF DESCRIPTION OF FIGURES

Features and advantages of the present invention will become apparent from the following description of embodiments thereof, by way of example only, with reference to the accompanying drawings, in which:

FIG. 1 is a schematic diagram of a system for monitoring personal activity, in accordance with an embodiment of the present invention;

FIG. 2 is a schematic diagram of a computing system which may be utilised to implement the system of FIG. 1;

FIGS. 3(a) and (b) show devices in accordance with an embodiment of the invention, presenting user interfaces;

FIG. 4 shows a device in accordance of an embodiment of the invention, presenting a user interface;

FIGS. 5(a) and (b) show devices in accordance with embodiments of the present invention, presenting user interfaces;

FIG. 6 shows a device in accordance with an embodiment of the present invention presenting a user interface;

FIGS. 7 and 8 show user interfaces presented by embodiments of the present invention;

FIG. 9 shows a user interface presented by an embodiment of the present invention,

FIG. 10a,b,c,d are example user interfaces which including interfaces which may be generated automatically depending on context and/or dimension data associated with user activity data;

FIG. 11 is a further user interface which may be generated automatically;

FIGS. 12a, b, c, d show further user interfaces which may be generated automatically in accordance with context and/or dimension data;

FIGS. 13a, b, c, d show further user interfaces which may be generated automatically in accordance with context and/or dimension data;

FIGS. 14 to 21 show example user interfaces that may be generated by a search process in accordance with an embodiment of the invention, in order to enable searching of user activity data;

FIG. 22 is a schematic diagram of a system in accordance with a further embodiment of the present invention;

FIG. 23 is a illustration showing a instructor user interface which may be generated in accordance with an embodiment of the present invention, and

FIG. 24 shows a further instructor user interface which may be generated in accordance with an embodiment of the present invention, and

FIG. 25 shows a further instructor user interface which may be generated in accordance with an embodiment of the present invention, and

FIGS. 26 to 36 are further interfaces which may be generated in accordance with embodiments of the present invention.

### DETAILED DESCRIPTION OF EMBODIMENTS

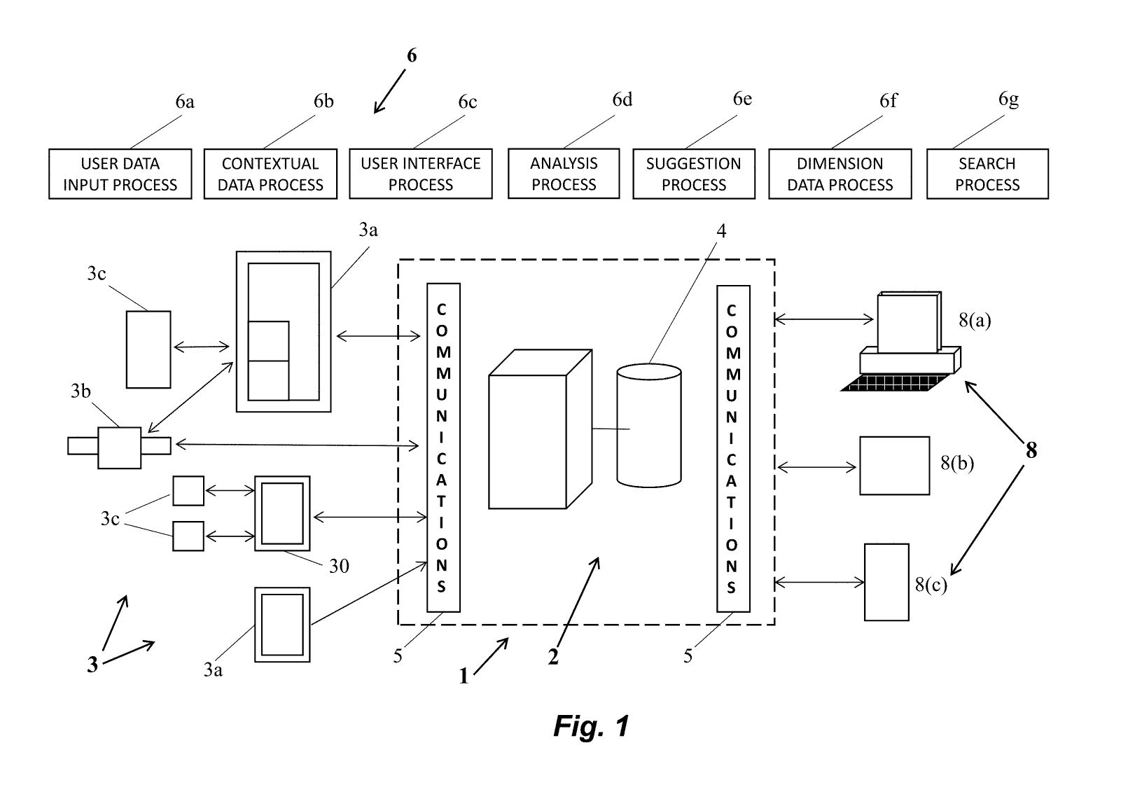

FIG. 1 illustrates a system for monitoring personal activity, generally designated by reference numeral 1. In this embodiment, the system comprises computing devices 2 and 3. Computing device 2 comprises a server computing apparatus including one or more processors, memory, an operating system for supporting computer processes and a database 4. Communications interfaces 5 are provided for communicating remotely. Note that the server computing apparatus 2 may be housed in the Cloud.

Computer devices 3 comprise mobile devices which may be operated by clients/users. The devices 3 may comprise mobile devices such as smartphones, tablets 3(a), wearable devices, such as smart watches 3(b) and any other devices 3(c). The devices 3 comprise computer processors, memory and operating systems for supporting computer processes.

A plurality of computer processes 6 are supported by the system 1.

The computer processes comprise a user data input process 6(a) and a contextual data process 6(b). The user data input process 6(a) is arranged to receive user input activity data, input to a device 3 via a user interface (see later). Contextual data process 6(b) is arranged to receive contextual data generated by a contextual data generating device and to associate the contextual data with the user activity data. Contextual data generating devices may include smartphones 3(a), smart watches 3(b) and other devices 3(c) which are arranged to generate contextual data.

The computer processes 6 may be supported by the server 2, and devices 3 may have remote access e.g. web browser access. Alternatively, the computer processes maybe distributed over the server 2 and devices 3. For example the user data input process 6(a) and contextual data process 6(b) may be supported by the devices 3, and other processes 6 may be supported by the server 2. Devices 3 may comprise native apps forming the computer processes and server 2 may support other applications of the system 1.

Further computing devices 8 may be provided for access to the system 1, to administer the system. Devices 8 may comprise laptops or PC's 8(a) tablets 8(b) smartphones 8(c) or any other device.

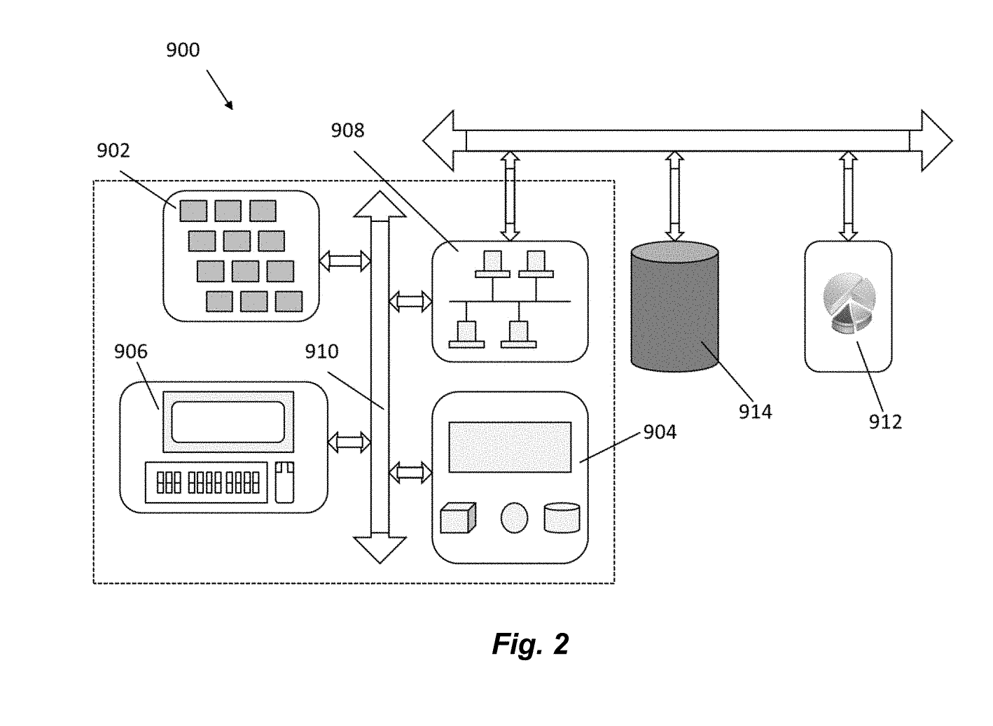

FIG. 2 is a schematic diagram of a computer system which may be utilised to implement the system of the embodiment of FIG. 1. Variations of the illustrated computer system may also be utilised to implement devices 3.

The computer system 900 may be a high performance machine, such as a supercomputer, a desktop workstation or a personal computer, or may be a portable computer such as a laptop or a notebook or may be a distributed computing array or a computer cluster or a networked cluster of computers. In the embodiment of FIG. 1, the computer 2 is a server computer. The invention is not limited to this arrangement. The computers may include any types of system discussed above. In an embodiment, the servers may be “virtual” servers implemented in the “Cloud”.

The computer system 900 comprises a suitable operating system and appropriate software processes for implementation of embodiments of the present invention.

The computer system 900 comprises one or more data processing units (CPUs) 902; memory 904, which may include volatile or non-volatile memory, such as various types of RAM memories, magnetic discs, optical disks and solid state memories; a user interface 906, which may comprise a monitor, keyboard, mouse and/or touch-screen display; a network or other communication interface 908 for communicating with other computers as well as other devices; and one or more communication busses 910 for interconnecting the different parts of the system 900.

The computer system for implementing embodiments of the invention is not limited to the computer system described in the preceding paragraphs. Any computer system architecture may be utilised, such as standalone computers, networked computers, dedicated computing devices, handheld devices or any device capable of receiving processing information in accordance with embodiments of the present invention. The architecture may comprise client/server architecture, or any other architecture. The software for implementing embodiments of the invention may be processed by “cloud” computing architecture.

In the embodiment of FIG. 1, the software processes 6 are shown implemented as separate modules. The invention is not limited to this. The software may be implemented in any convenient software architecture, routines or sub routines, or any other architecture that can implement the functionality described in this description.

Referring again to FIG. 1, the database may receive and store user data and contextual data input via processes 6(a) and 6(b), for many users.

The computer processes also comprise a user interface process 6(c) that provides various interfaces to users to present with them with information and also to facilitate the input of user data.

An analysis process 6(d) and suggestion process 6(e) is provided for analysing user data and contextual data and providing suggestions back to user devices 3. Suggestions may provide useful information or proposals to facilitate a users well-being, and/or to provide other information.

Operation of the system of this embodiment will now be described in more detail with reference to the following examples.

This embodiment provides an arrangement which enables an intuitive process for a user to create and automatically add context to life's “moments”, combining in one intuitive note creation process user and contextual data from contextual data generating devices and processes, such as health applications, weather applications, location, time and other inputs.

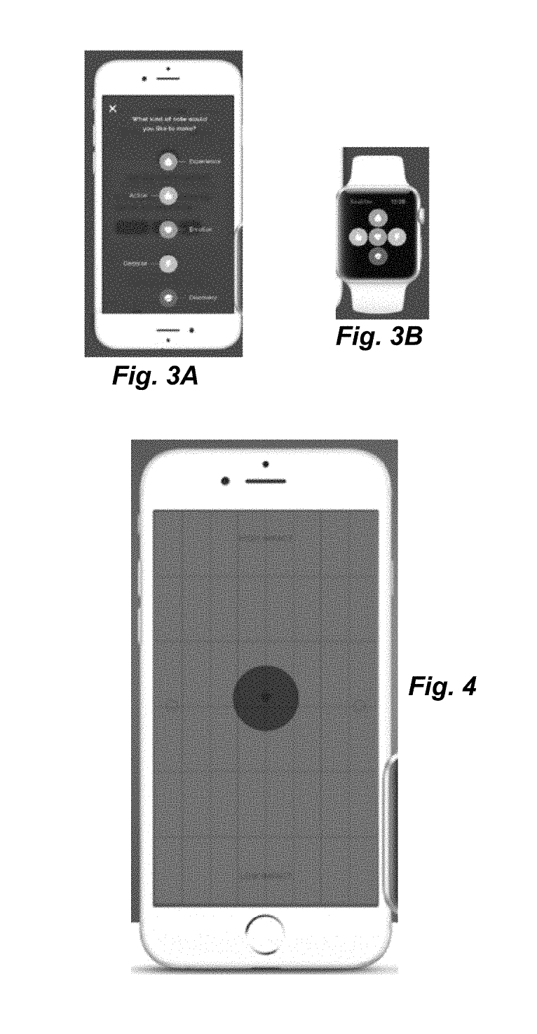

FIG. 3(a) and FIG. 3(b) show interfaces for user devices 3, in the form of a smartphone interface 3(a) and a smart watch interface 3(b). The user interface process 6(c) is arranged to generate these interfaces so that the user can input data.

In this embodiment, the system also comprises a dimension data process. The dimension data process enables the association of dimension data with user input activity data. In this embodiment, the dimension data may include “categories”. Dimensions may also be selected and input by users. The user interface process may enable selection of dimensions and/or input of dimensions.

The user may choose a dimension, such as a category: Experience, Action, Emotion, Decision, Discovery as shown FIG. 3(a). This gives a category to the entry to be made by the user. These categories are shown as icons on the smart watch 3(b).

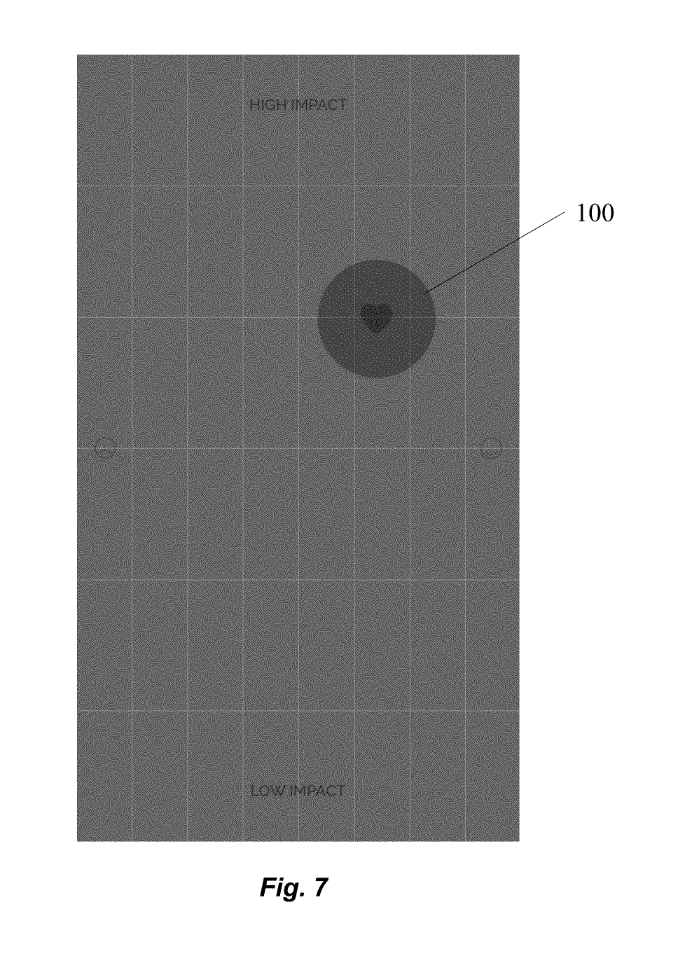

Referring to FIG. 4, on the smartphone interface a matrix is presented which enables the user to enter the “impact” and the “feeling” associated with the life experience. This is shown in more detail in FIG. 7. The user is able to move a cursor 100 across the matrix to the location that they determine best represents the impact and feeling that they are experiencing at this particular moment/event. The matrix in this embodiment contains 48 cells to enable precise definition of the user perception. The cursor beats like a heart to symbolise the pulse of life.

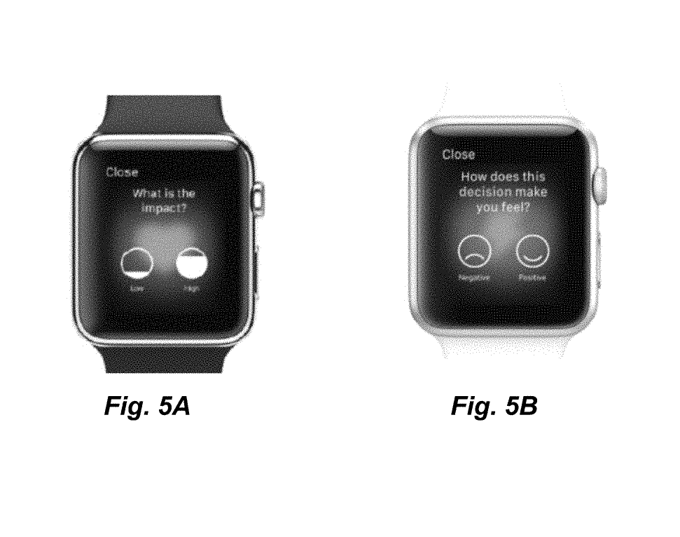

FIGS. 5(a) and 5(b) show interfaces generated to smart watches, which enable the user to enter their impact and feeling.

A “Score” may be calculated from the matrix input and other factors, and the Score may be presented to the user. These may include an Impact Score, 0 to 100, a Feeling Score, 0 to 100 and a Well-being Score which may be a weighted combination of the Impact Score and Feeling Score (range −50 to +50), for example.

Devices and computer processes are available which can obtain physical data, including physical data about a user (e.g. medical data) and also environmental data about the environment (temperature, weather). In this embodiment, the contextual data process 6(b) obtains this data from the devices that generate it and logs this data in the system 1, together with the user data input. The Dimension data (e.g. category) may also be stored associated with the user activity data which has been input.

The contextual data may be obtained from any device. The smartphone that the user is using for user input may include applications, such as weather applications, fitness applications, and other applications. Smart watches and other fitness devices may be able to obtain blood pressure, heart beat and number of “steps”. There are also other devices e.g. bicycle computers, that can monitor heart rate, distance traveled, etc. The contextual data process can interface with these devices and processes and obtain the contextual data. The data may include, but is not limited to;

Date; time; location; heart rate (BPM); weather conditions; weather temperatures; steps; distance traveled; calories burned; amount of sleep, and other contextual data.

The system associates contextual data with the user data input. For example an impact or feeling label may be automatically associated (e.g. High Impact a positive emotion). An impact score may be calculated A feeling score and a well-being score may be calculated.

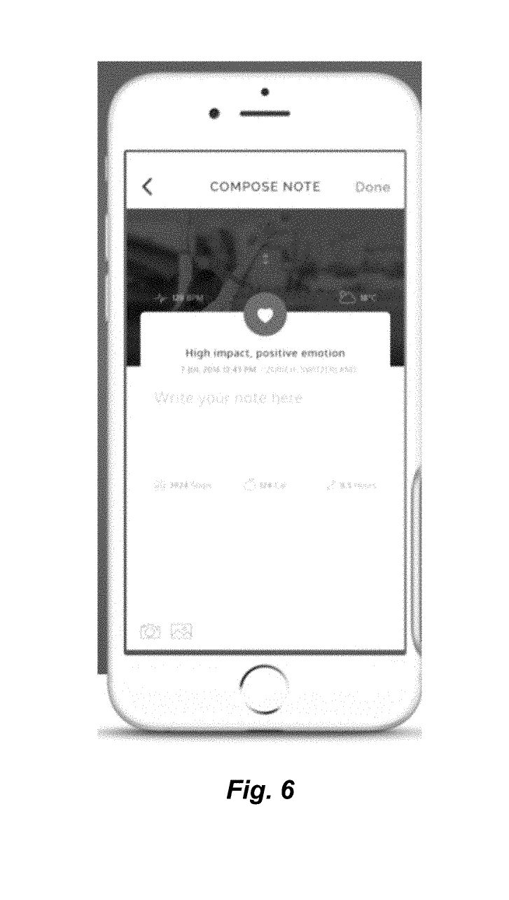

The user data can comprise a note to be input as user data input (see FIG. 6). These notes can be dictated or typed.

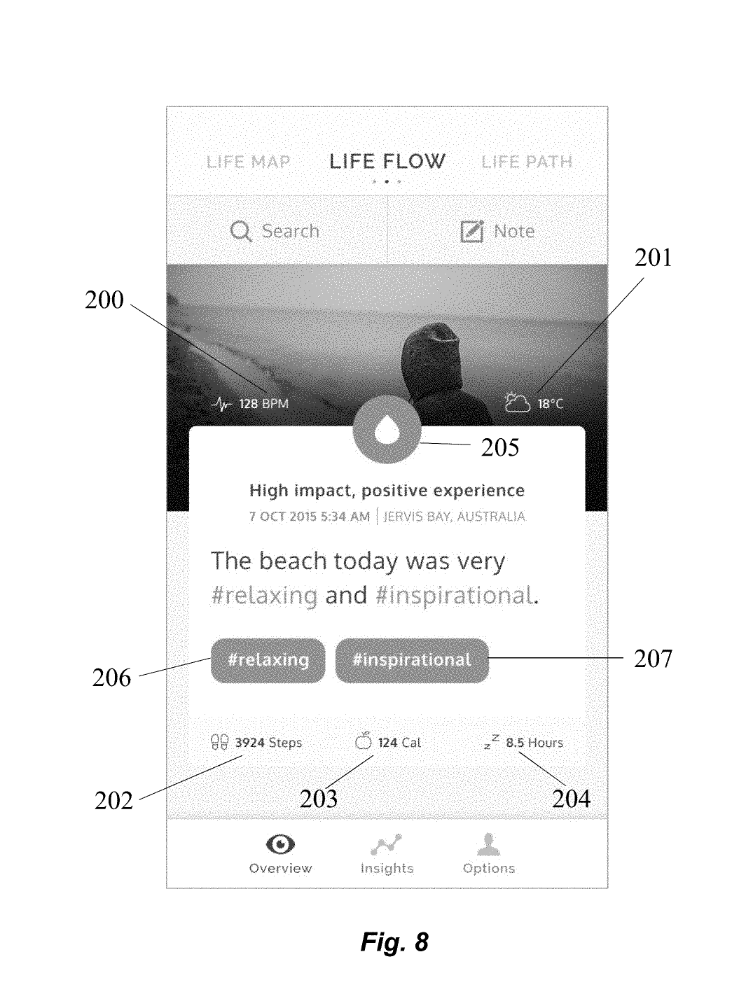

The user data input and contextual data are associated and can be presented back to the user. See FIG. 8. (The “results” interface). When the user views a note or group of notes that they previously entered, for example, the contextual data is also displayed (see items 200, 201, 202, 203, 204). They can therefore view the contextual data in relation to any user input data they may have made. This may be displayed in a number of ways, FIG. 8 being merely one way.

In this embodiment, an analysis process 6(d) and suggestion process 6(e) is arranged to analyse the user input data on activity and also analyse the contextual data which is obtained. In response to the analysis, a suggestion process provides suggestion hashtags or suggestions back to the user devices 3 for display to the user.

FIG. 8 also shows a dimension which is allocated to this example (in this case, Experience 205). FIG. 8 also illustrates another feature of this embodiment, that a user can select their own dimensions that can be subsequently searched by. In this case the user has selected #relaxing and #inspirational (ref numerals 206 and 207). The user is therefore able to develop options to provide a more granular and detailed look into their life.

Each user has a personal experience and only receives the relevant suggestions for them based on individual or combined contextual data, dimension data and scores:
Topics
Heart Rate (BPM)
Weather Conditions
Weather temperature (Celsius or Fahrenheit depending on the region)
Steps
Calories burned
Sleep
Impact score
Feeling score
Well-being score
Over a period of time

Each suggestion is also sent at the relevant time depending on the location/timezone of the user.

All these criteria can be combined. Example:

A suggestion will be sent to:
All the users
Who have created over the last 7 days
More than 3 notes about #Work
For which their well-being was on average: negative (between −50 and −12 in a scale ranging between −50 and +50)
Suggestion sent=Struggling at work?

OR
All the users
Who have created over the last 30 days
More than 5 notes on an active day (High number of steps OR high heart beat OR high calories burned)
For which their well-being score was on average: positive (between 12 and +50 in a scale ranging between −50 and +50)
Suggestion sent=Activity is good for your well-being.

OR
All the users
Who have created since they started using the app
Notes when it was raining or when there were thunderstorms
For which their Mood score was on average: negative (between 0 and 30/100)
Suggestion sent=Rainy days are bad for your mood!

OR
All the users
Who have created since they started using the app
Notes after a short night sleep
For which their Mood score was on average: negative (between 0 and 30/100)
Suggestion sent=Increasing your Sleep hours is important for you to keep a positive attitude.

Any suggestions may be made, depending on context and user input. The embodiment is not limited to the examples given above.

Input user activity data and contextual data may be stored in the database 4 or any other parts of the system. It also may be retrieved, so the user can review their input activity data historically. In an embodiment, a user is able to retrieve associated user activity data by entering contextual data. For example, contextual data may include a time, a date and the user can retrieve associated user activity data for that time and date. Any other contextual data can be used to retrieve user activity data. For example, user activity data could be retrieved based on weather (“what was I doing on dull days”); health data (“what was I doing when my heart rate was high”), or any other contextual data.

User activity data may also be retrieved based on associated dimension data. For example, a user may search using the “Action” dimension (see FIG. 3). All user activity data entered under the action dimension may be retrieved. Combinations of contextual and dimension data can be used to search. For example, all activity data under the action dimension that was entered on rainy days could be searched and retrieved. Search process 6g (FIG. 1) enables data to be retrieved based on search functionality using dimension data and context data. In this embodiment, this is known as an “Explore” feature. The Search/Explore process can be used by a user to draw down with fine granularity on their user activity data, to enable them to explore their life data.

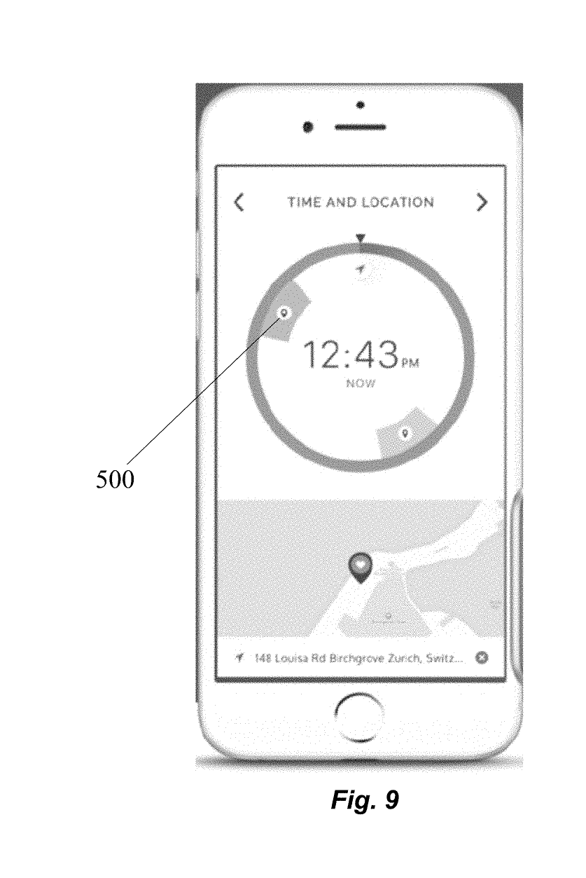

Referring to FIG. 9, an interface generated by an embodiment which enables entry of user activity data for times in the past, will now be described. In this embodiment, the user data input process may enable a user to designate a time in the past for which they wish to enter user activity data. For example, this enables an additional step in the note creation flow between the categories selection screen and the perception matrix. The user merely has to select one of the time periods that appear on the screen e.g. reference numeral 500. The system will automatically then retrieve contextual data. For example, it may retrieve: location, heart rate, weather conditions, weather temperature; steps, calories burned, sleep and any other location data. This contextual data will be as of the time in the past that is designated by the user input. If they wish, the user can then enter user input activity data for that time and it will be associated with the retrieved contextual data.

The Analysis process and suggestion process (6d and 6e) are implemented utilising software filters, software grouping, and templates enabling suggestions. The analysis and suggestion process comprises a sophisticated “personalisation and suggestions” Engine. The suggestion process 6e can also work with the user interface 6c to present and “shape” user interfaces to direct the user focus on particular areas.

The technical components and implementation of a personalisation and suggestion engine and automated adjustment of user interface will now be described.

Components

1. Bespoke Sophisticated Personalisation and suggestions Engine (back end) to target micro—audiences using their real-Life data to deliver highly personalised suggestions:

a. Sophisticated Filtering Engine

b. Tables (Database Model)

c. Suggestion templates

d. Merge fields

2. Bespoke features in the app (front end) that allow the user to visualise their “Life Autofocus” suggestions (suggestions automatically provided to the user interface) and user “Life Autofocus” to automatically shape their dashboards (Automatically adjust and/or present user interfaces)

a. A suggestions screen to display Life Autofocus suggestions;

b. A call to action—Autofocus button—visible on the Life Autofocus which automatically shapes users' dashboards using the explore (search) functionality

c. A sophisticated “Explore” (search, using filtering technology), functionally driven from the personalisation engine:

A. Sophisticated Filtering Engine

The filtering engine uses different layers that allow an extremely fine segmentation.

Filters

Filters are unit queries on the database model which can be used in a rule.

Groups

A group allows to apply an ‘OR’ operator between several filters.

Rules

A rule is a combination of filters and groups that allows to narrow down the results. It works like a funnel. The sequence of filters and groups determine the results.

In the example above, the rules engine will target all RealifeChange users that have created in the last 7 days at least one note about their emotions that contributed to the user's positive well-being.

List of the Main Filters

B. Tables

There are several tables in the model we can query on. The main ones being:
Users
Notes
Hashtags
Life Activity

### Example: Notes Table

C. Suggestions Templates

Suggestions can be set to real-time or batch. If sent in batches, the system will send suggestions to the targeted audience, taking into account their time zone to avoid sending suggestions to users in the middle of the night.

An autofocus call to action with parameters can be added to the suggestion (in the notification template). These parameters will be carried across to the front end (the app) to automatically shape the users' dashboards using the Explore (filtering) functionality.

Some Examples of Suggestion Templates

This suggestion will be sent to 30 users who have expressed difficulties (negative feelings) about their work. It will give the well-being status (very positive, positive, negative or very negative) to the user using a merge field.

This suggestion will be sent to the 7 users who have expressed satisfaction being with their friends (positive feelings). It will give the mood and impact scores to the user using merge fields.

This suggestion will be sent in real-time to the 240 users who have created 12 notes within one hour on 1 Nov. 2016. This suggestion will give the average well-being score of the user using merge fields.

D. Merge Fields

Merge fields allow to insert user data into the suggestions to create highly personal content that will be sent to the user.

List of the Main Merge Fields

Keys

Notes

{{total_notes}}

{{avg_notes}}

Latest Note

{{steps_latest_note}}

{{temperature_latest_note}}

{{sleep_time_latest_note}}

{{feeling_score_latest_note}}

{{impact_score_latest_note}}

{{wbs_latest_note}}

{{suburb_latest_note}}

{{category_latest_note}}

{{weather_latest_note}}

{{date_latest_note}}

{{time_latest_note}}

Average

{{avg_steps}}

{{avg_temperature}}

{{avg_sleep_time}}

{{avg_feeling_score}}

{{avg_impact_score}}

{{avg_wbs_score}}

Topics

{{topics_latest_note}}

Most Used

{{most_used_category}}

{{most_used_suburb}}

{{most_used_date}}

{{most_used_weathter}}

Most Used

{{most_used_topic}}

User Activity

{{goal}}

{{time_spent}}

Average WBS with Status

{{avg_wbs}}

{{staus}}

Component 2: Bespoke Features in the App (Front End)

Composed by:

A suggestions screen to display Life Autofocus suggestions;

A call to action—Autofocus button—visible on the Life Autofocus suggestions which automatically shapes users' dashboards using the explore functionality.

A sophisticated “Explore” (filtering) functionality driven from the personalisation engine;

Description:

A user receives a push notification (suggestion) in the RealifeChange app from the personalisation/suggestion engine in the back-end.

They visualise the suggestions with personal data included (merge fields).

They only receive this suggestion if its content is relevant for them in relation to the real life data that they have entered as notes in the app.

The call to action—Autofocus button—contains the filtering parameters, the option to open the Well-being score window and the name of the dashboard to which the user will be automatically directed that has been configured in the notification template (in the back end).

User Flow:

The User visualises their personalised Life Autofocus suggestion.

The user taps on the call to action—Autofocus button.

The user is automatically directed to the dashboard selected in the notification template in the back-end

The well-being score window will be shown to the user if the option has been ticked in the notification template in the back end.

The app will automatically apply the right filtering parameters (using the parameters that have been defined in the notification template in the back end).

The filtering feature—explore—is automatically activated. It filters the data using the parameters selected in the notification template in the back-end). The user can open the Explore feature (filtering) to see which filters have been applied.

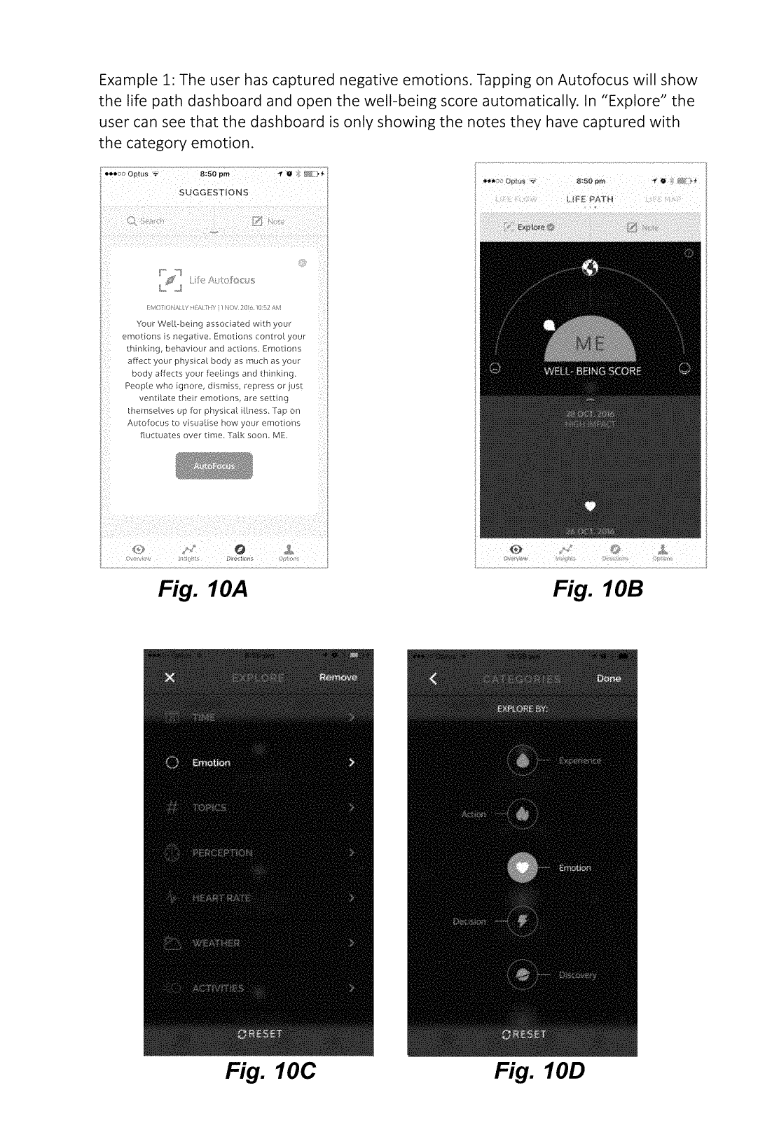

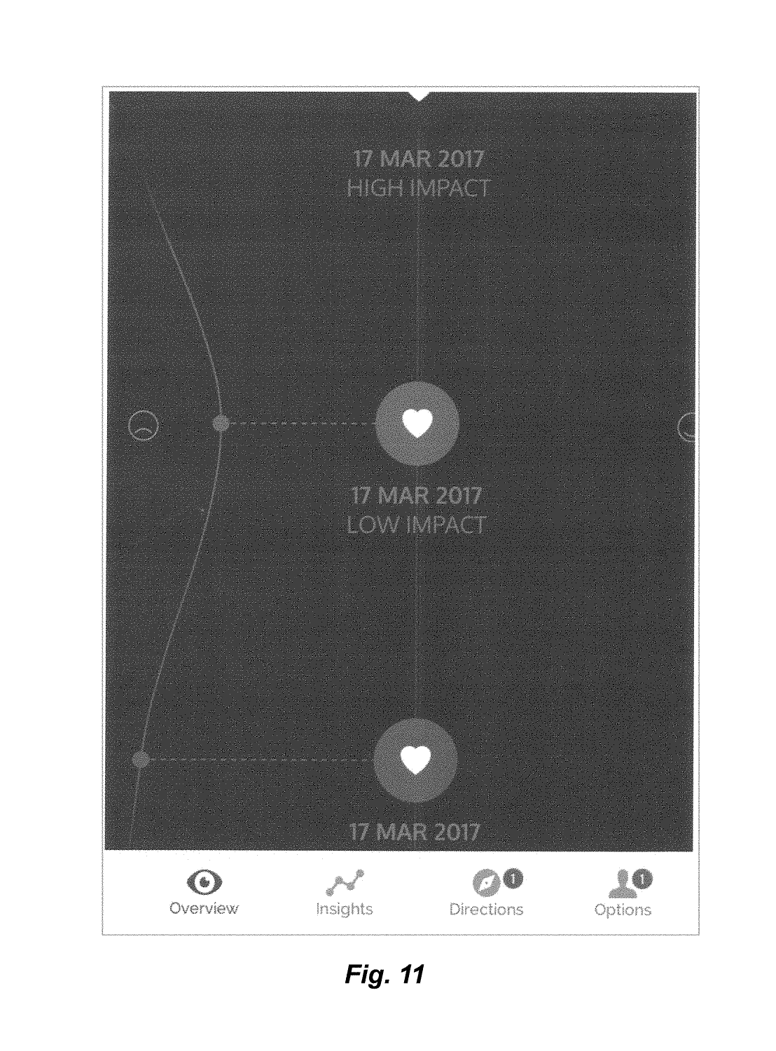

An example of the operation of the analysis process and suggestions process, 6d, 6e, to adjust the user interface will now be described with reference to FIGS. 10 and 11.

In this example, the user captures negative emotions. A suggestion (FIG. 10a) is provided to the user together with an interface (auto focus button 50). Tapping on autofocus 50 will show the lifepath dashboard (FIG. 11) and open wellbeing score automatically (FIG. 10b). In “explore” (FIGS. 10c and 10d) the user can see the dashboard is only showing the notes they have captured with the category “emotion”. The user can therefore review all their notes associated with negative emotion to see what the problem is and see if they can fix it. The search process is implemented automatically by the auto focus action to focus the user on these interfaces.

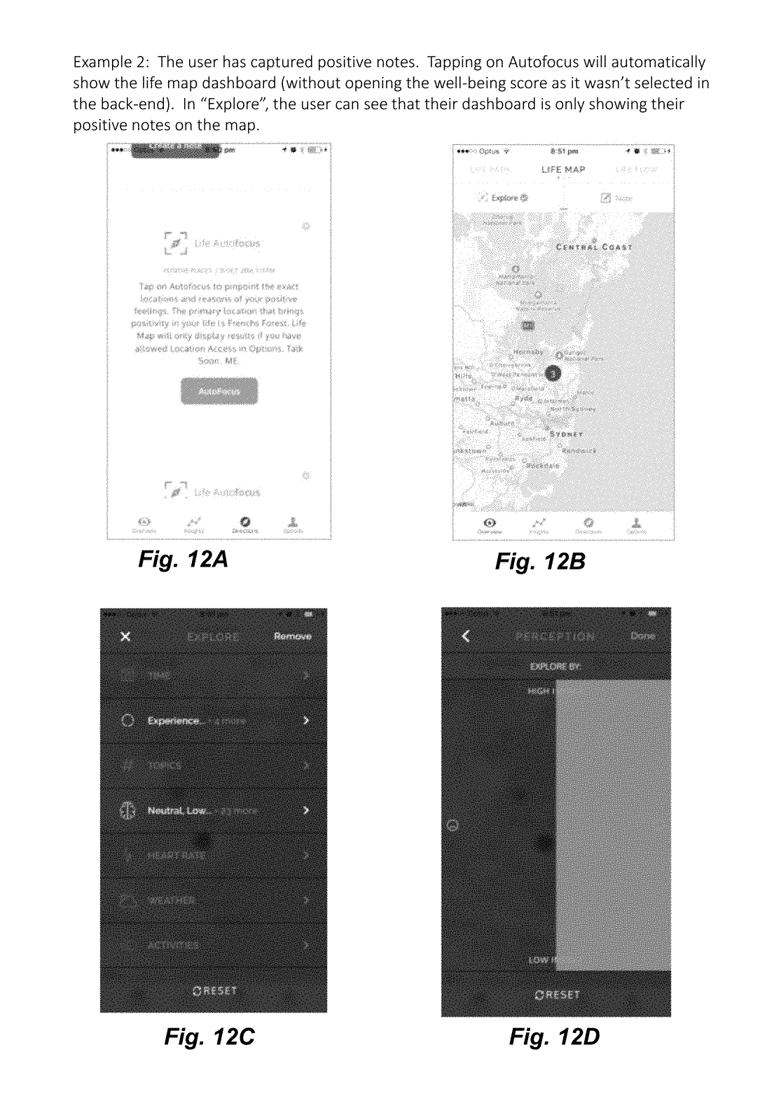

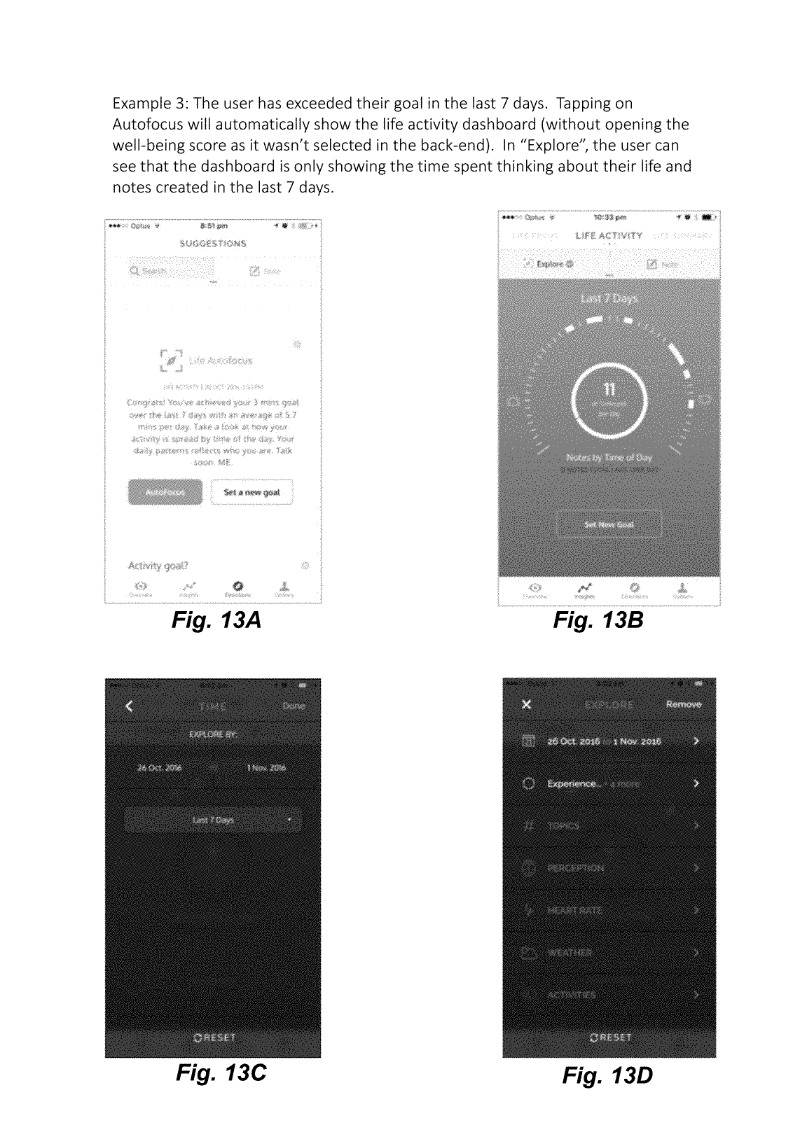

In example 2, FIG. 12, the user has captured positive notes. An automated suggestion (FIG. 12a) is sent to the user. Tapping on the auto focus button 50 shows the life map dashboard (showing where the user has captured positive emotion). The wellbeing score isn't open as it wasn't selected by the analysis and suggestion process. In Explore, the user can see their dashboard is only showing their positive notes on the map (FIGS. 12c and 12d).

In example 3, the user has exceeded their goal in the last 7 days. Tapping on auto focus 50 automatically shows life activity dashboard (13b). In Explore the user can see that the dashboard is only showing their time_spent thinking about their life and notes created in the last 7 days (13c and d).

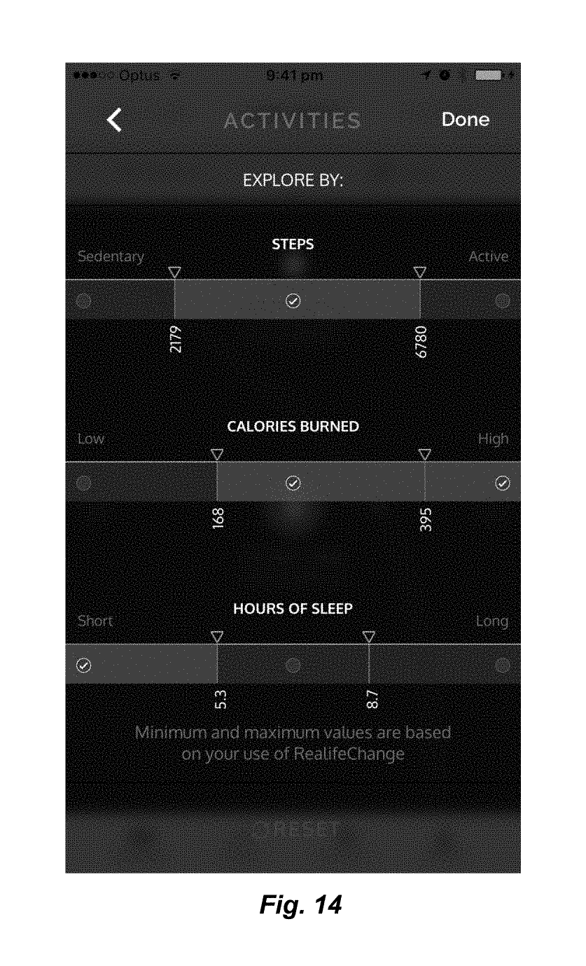

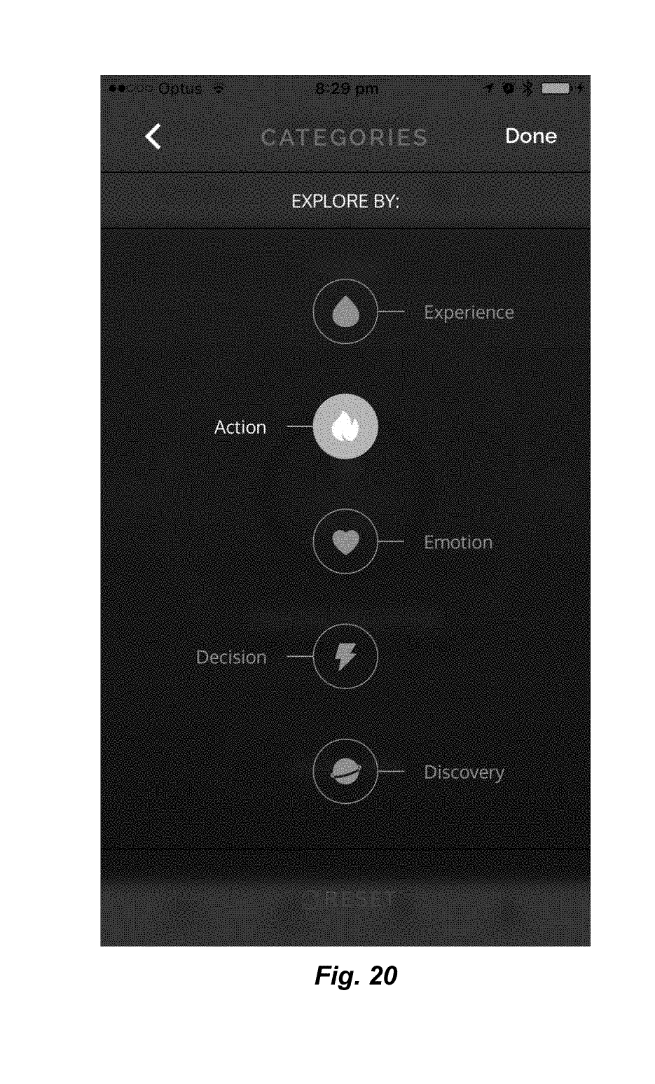

As discussed above, a search process 6g is provided in this embodiment which enables a user to Explore what has been happening in their life relating to the user activity data that has been input by them. This Search/Explore functionality will now be described in more detail with reference to FIGS. 14 through 21.

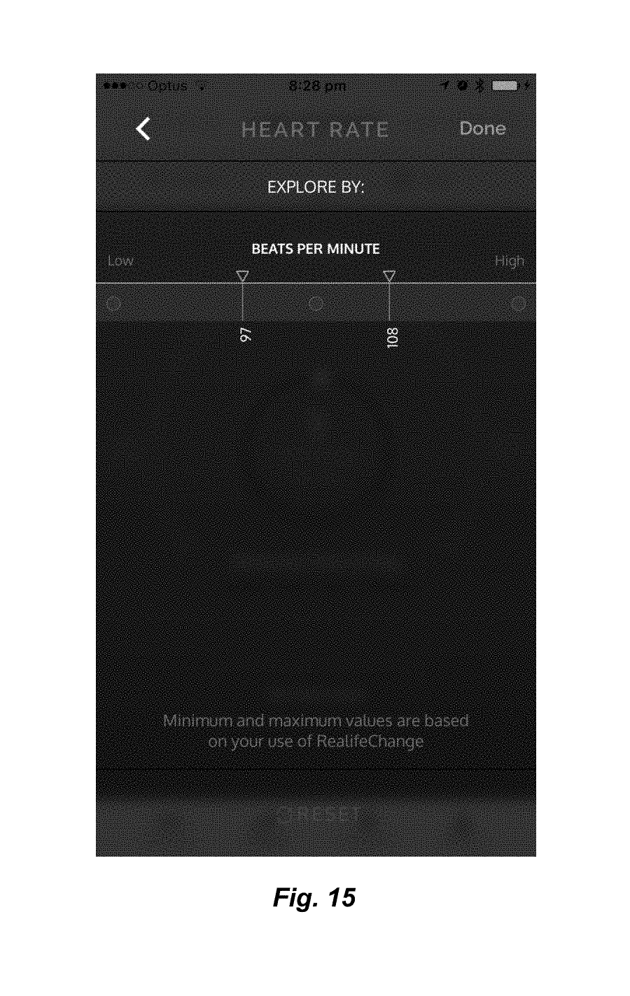

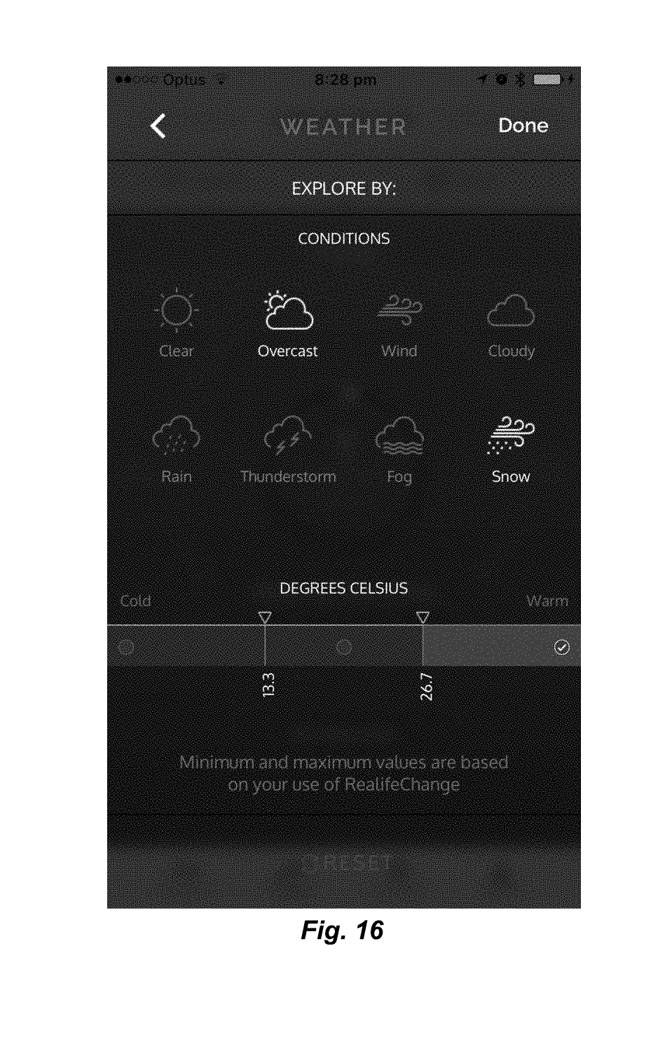

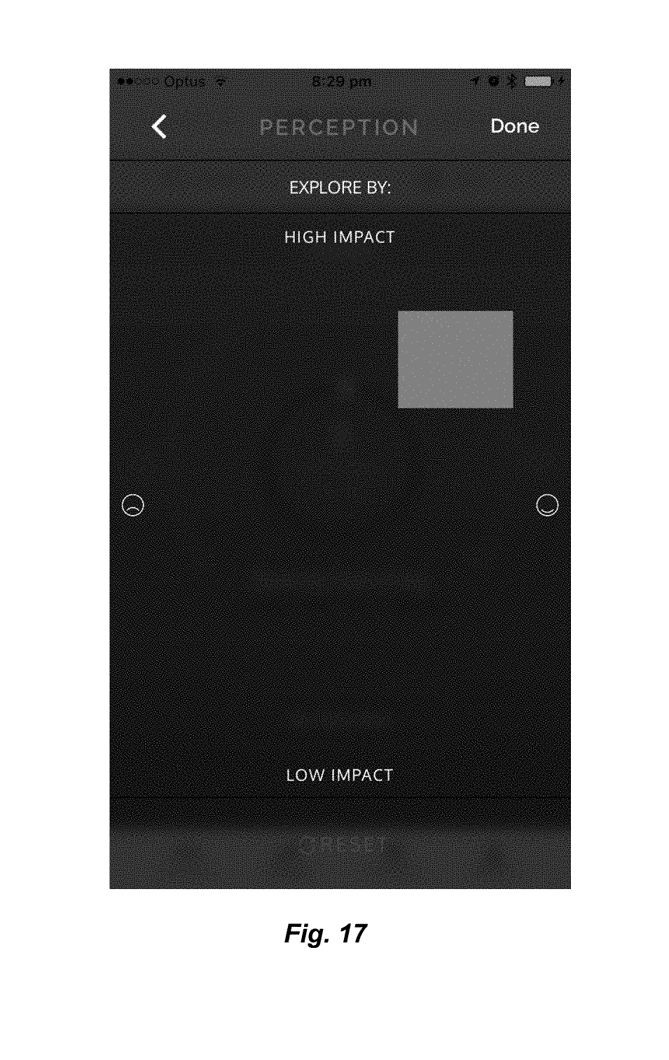

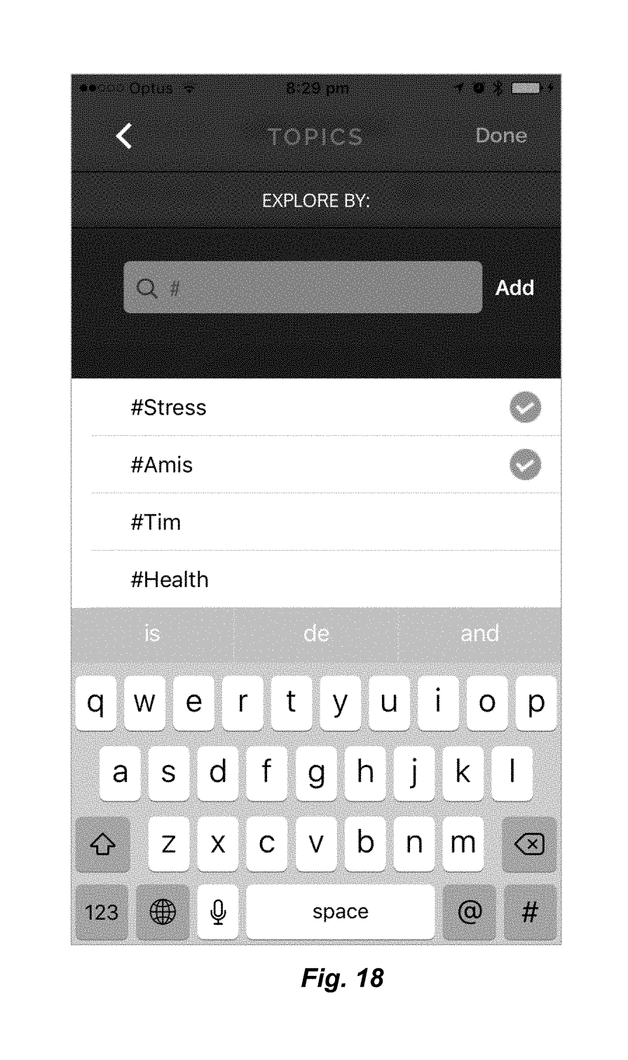

The user may search/explore by any dimension or context data that has been associated with activity data. For example, they may explore by Steps, Calories burned, hours of sleep, heart beat (FIGS. 14 and 15). They may explore by weather conditions (FIG. 16). For example, any notes they input when the weather was overcast may be returned to them so they can analyse them. Similarly temperature (FIG. 16). They may by explore by the dimension “high impact” (FIG. 17). See the shaded square in FIG. 17 which will bring back all user activity data associated with this impact level.

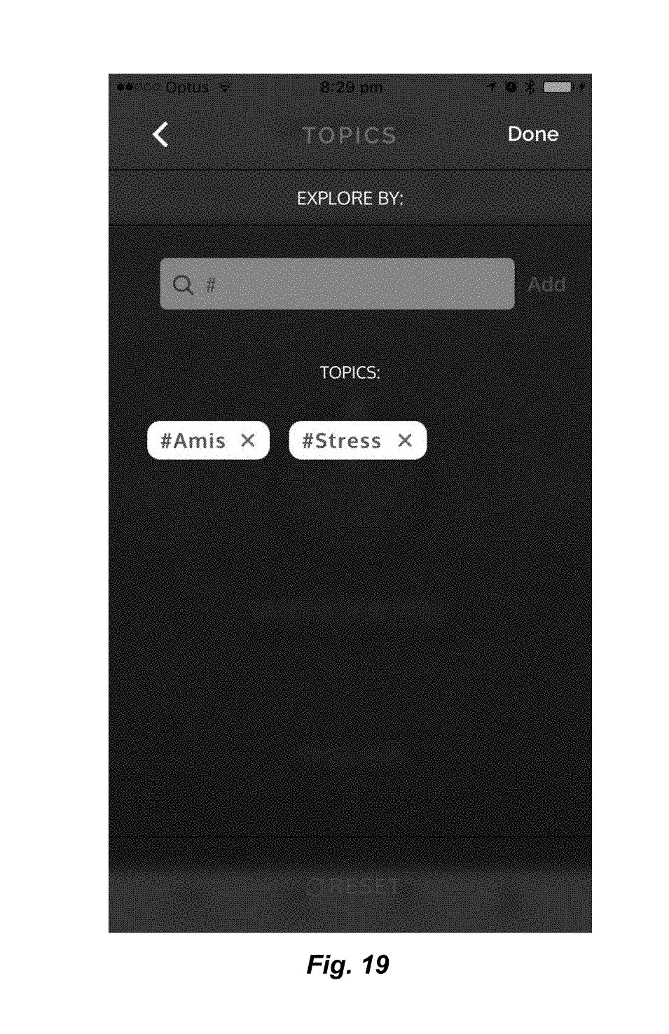

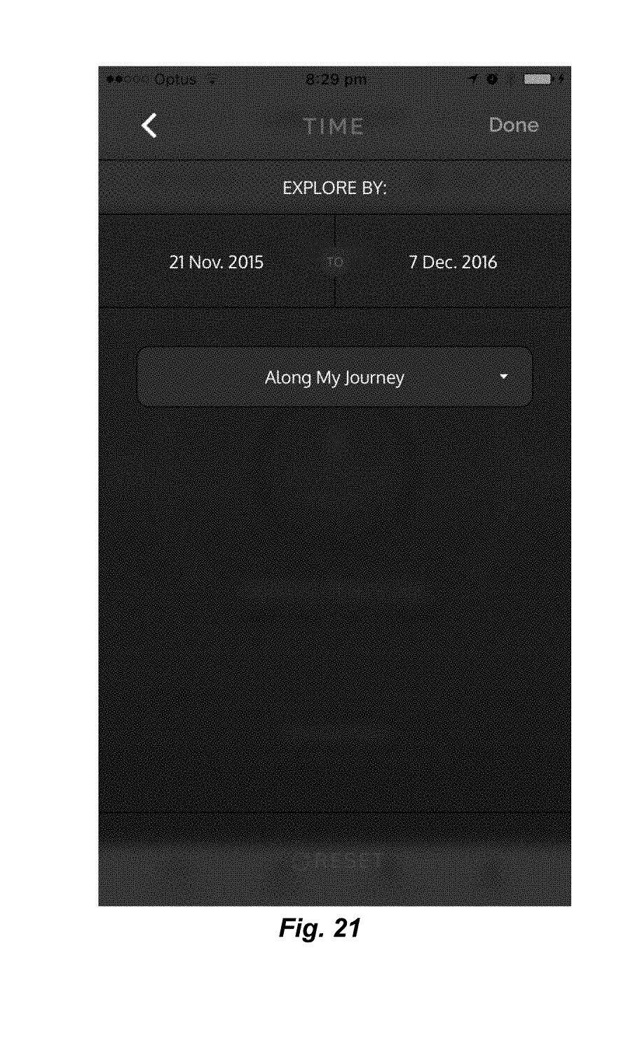

They may explore by “topics” they have themselves selected as hashtags (FIG. 18 and FIG. 19) they may explore by time (FIG. 21).

The user may explore by any dimension and context. This way the user can step back through time in many dimensions and contexts in order to analyse what has been going on in their life. Search functionality is implemented by filters and groups as discussed above.

An “impact”, “mood” and “wellbeing” Score may be calculated depending upon dimension and/or context selected. A user, for example, may decide they want to explore by “experience” in the last seven days. A mood score is calculated from the matrix interface entries they have made under “experience” dimension in the last seven days.

In this embodiment, each filter in an explore interface (see FIGS. 14 through 16) may be segmented. In this embodiment three segments are shown, but there may be more or less. The user can select which segment to explore by. For example in FIG. 15, the user has selected a middle segment of Steps to explore by (step 2179 to 6780). They have also selected the middle segment of calories burned and the first (low) segment of Hours of Sleep.

The minimum and maximum values in the segmentation in this embodiment are based on users' actual use. For example 6,000 steps might be a maximum, but a particular person may do 10,000 steps a day. The scale is varied depending on the user input. Further the user can slide to change the segments. Minimum steps might be from 100 to 5,000 by default, but the user might want to analyse from 100 to 1,000. They can do it by moving the sliders (arrows in FIG. 15). The ability for the search process to enable a user to focus on particular aspects of their life by context data and/or dimension data, is a powerful tool which enables the user to analyse their life when they are utilising the system.

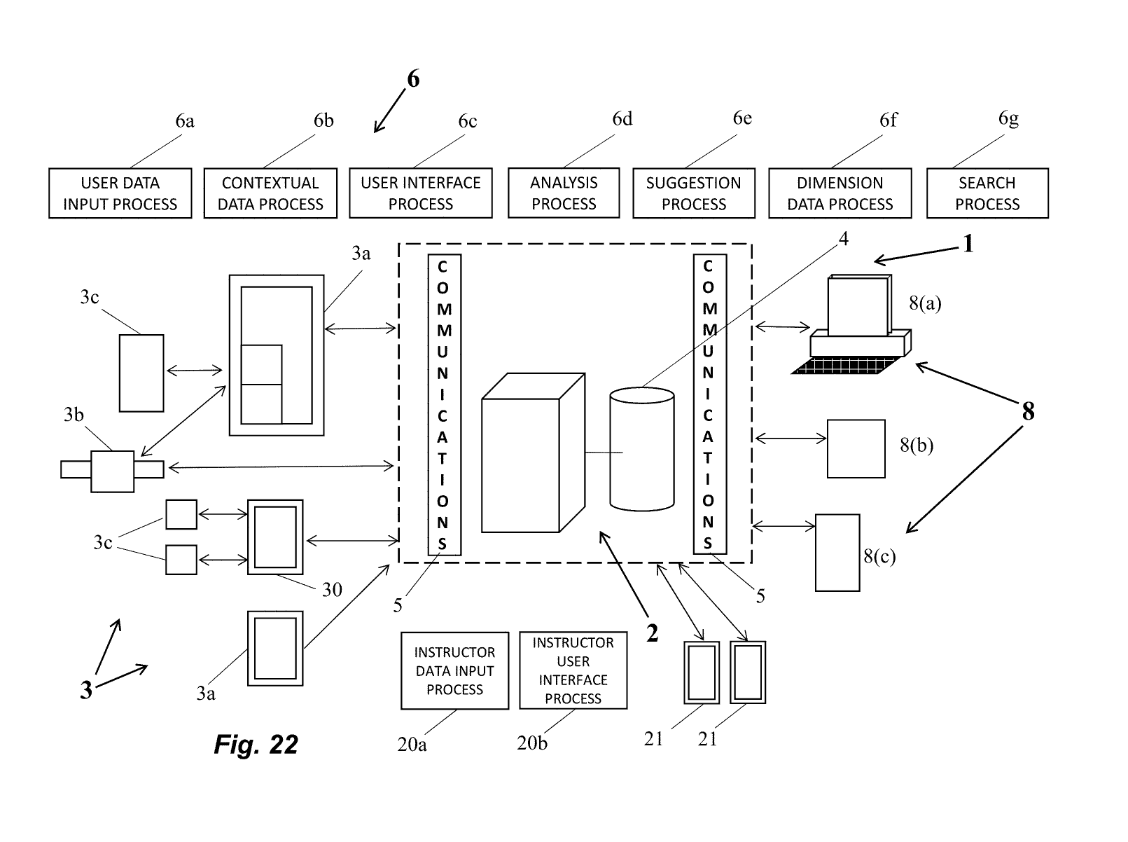

A further embodiment of the present invention will now be described with reference to FIGS. 22 to 25.

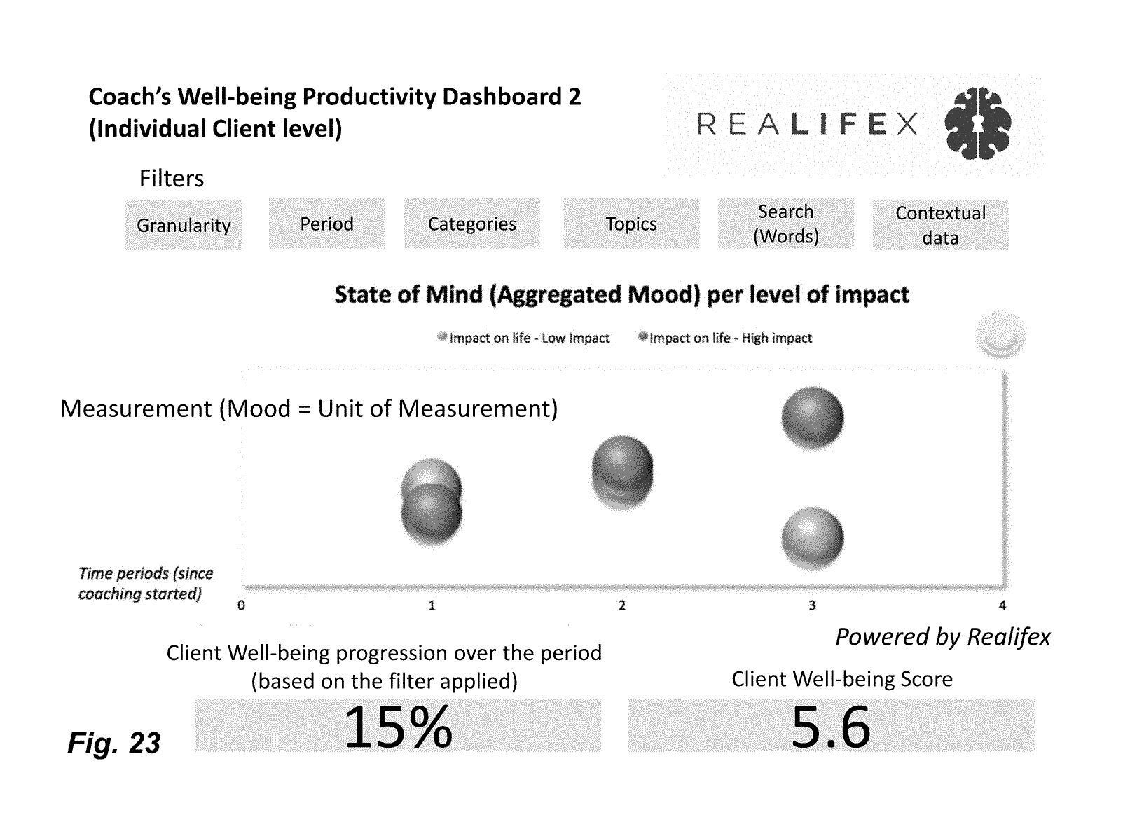

In the above embodiment, the system itself provides the artificial intelligence for processing the user data activity and providing automated suggestions to users. In the above embodiment, the system itself acts as the “life coach”. The embodiment of FIGS. 23 to 25, enables instructor access to the system. Instructors, such as life coaches, may therefore monitor the interaction of their clients (users) with the system and may interact with the users via the system. Rather than the current state of affairs, where a life coach and user can only get together infrequently, utilising this embodiment an instructor may have an insight into the ongoing interaction of the user with the system, and may be able to provide comments and interjections at appropriate points. The instructor will have access to all the data which is selected by the client for instructor access. Advantageously, instructors may handle multiple clients via the system. This is more convenient for the instructor, as well as more effective for the user. The user not only has the benefit of the AI of the system, but also can interact with a “real life” coach. The Instructors may see the latest updates (user activity data) from their clients in real time. They may visualise, analyse and comment on wellbeing insights in preparation for the next face to face session with the client, using the client contextual and dimension data, wellbeing scores, impact on their life by the dimensions. They may schedule or provide real time suggestions. They can schedule suggestions for later, for example.

Referring to FIG. 22, the same reference numerals have been used to denote the same components as the FIG. 1 embodiment. No further description will be given of these components.

In addition to these components, the system now also includes an instructor data input process 20a and instructor user interface process 20b. These enable instructors users (e.g. life coaches) to input data for clients and receive information (via the user interface process) 20b. Devices 21 of the instructor users may comprise smart phones, tablets, or any computing device. The devices 21 communicate with the server 2 and database 4 as previously described.

Instructor user input process 20a has a secure connection with the system. An application may be provided on the instructor user device 21, which enables the instructor data input process 20a and instructor user interface process 20b. The user data input process and user interface process enables the user to implement sharing their user activity data with an instructor user in a secure manner There are a number of technical features that need to be implemented to ensure connection security and other functions.

Connexion Security Between RealifePortal (the Instructor Interface and Processes) and RealiChange (the User Interface and Processes)

Upon sign up in RealifePortal (the instructor user app) with the email address and 8 digit password composed of upper and lower case letters and numbers, the system generates a unique 16-digit secure Access Code, composed of upper and lower case letters and numbers;

Once the coach accepts the terms and privacy policy, we prompt the access code on the screen;

c) The access code is then sent to the clients (users) via email;

d) The client signs into RealifeChange (clients' app) with their account protected by email and password, and in their Options screen in RealifeChange, they activate “Share life Data”. They copy/paste the Access Code in that screen e) In real-time, the client's life data appears in RealifePortal.

f) Note: The client can, at any time, inactivate “Share life Data” in RealifeChange. In real-time all the online data will be instantly removed from RealifePortal.

Any user activity data input via the process 6a “shared” with the instructor “coach” via the instructor user interface process 20b.

2. Real-Time Sync without Refresh

The system utilises a technology called Ember.js™ (Single page application). This technology enables to upload all pages of the app upon login and allow navigating in the app during the session without having to load any pages. No waiting time.

There are 2 complexities:

Complexity 2a): Clients can Activate/Inactivate “Share Life Data” at any Time OR Create/Delete Notes at any Time.

Processes are run in the background during the session to show or hide clients and notes in real-time WITHOUT any browser page refresh from the coach in RealifePortal. This creates a rich user experience as the coach will see their screens, dashboards, data on the screen change depending on what all their clients are experimenting in their life.

Complexity 2b): A Coach can Simultaneously Use Several Devices

A coach can change their plans (upgrade, downgrade, cancel, reactivate); can create, update or delete a private comment; create a suggestion. This information needs to flow in real-time between devices and update the screen WITHOUT any browser page refresh. The system runs processes in the background during the session to make this happen.

3. Send Suggestions

From RealifePortal, a coach can send suggestions to their clients who will receive push notifications (suggestions) directly in the RealifeChange app. The personalisation engine Life Autofocus is used to make the solution work. A suggestion can be sent to one or several clients. Can be sent in real-time or scheduled to a later date

A specific API has been developed to create a filter and a rule with the selected clients and create a suggestion template, with the name of the coach as a pre-defaulted subtitle. The admin console has been updated to separate suggestions that have been created by coaches from the Life Autofocus suggestions.

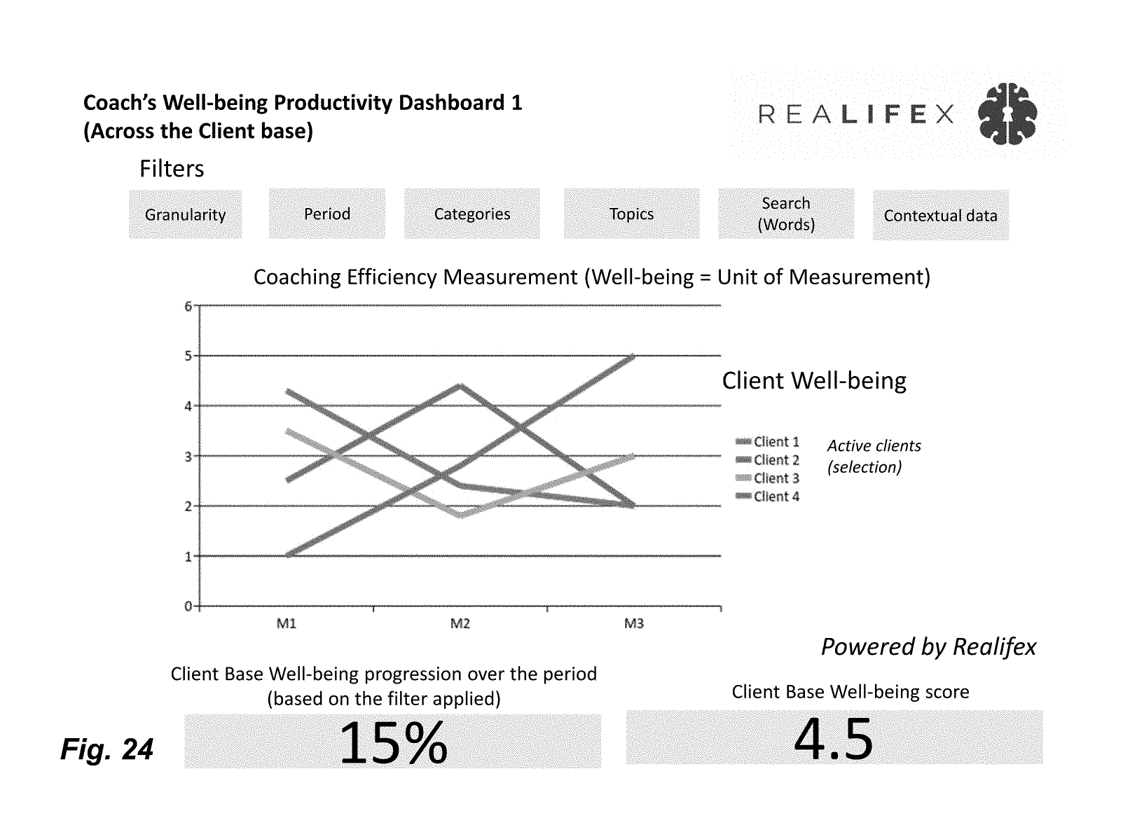

Utilising filtering on dimensions, context, etc, the instructor user interface can generate tools enabling instructor user to determine the efficiency of their coaching/mentoring. FIG. 24 shows a “mood” display which shows client wellbeing progression over a period determined by a filter.

FIG. 24 shows the progress (wellbeing) of a number of clients over time (months).

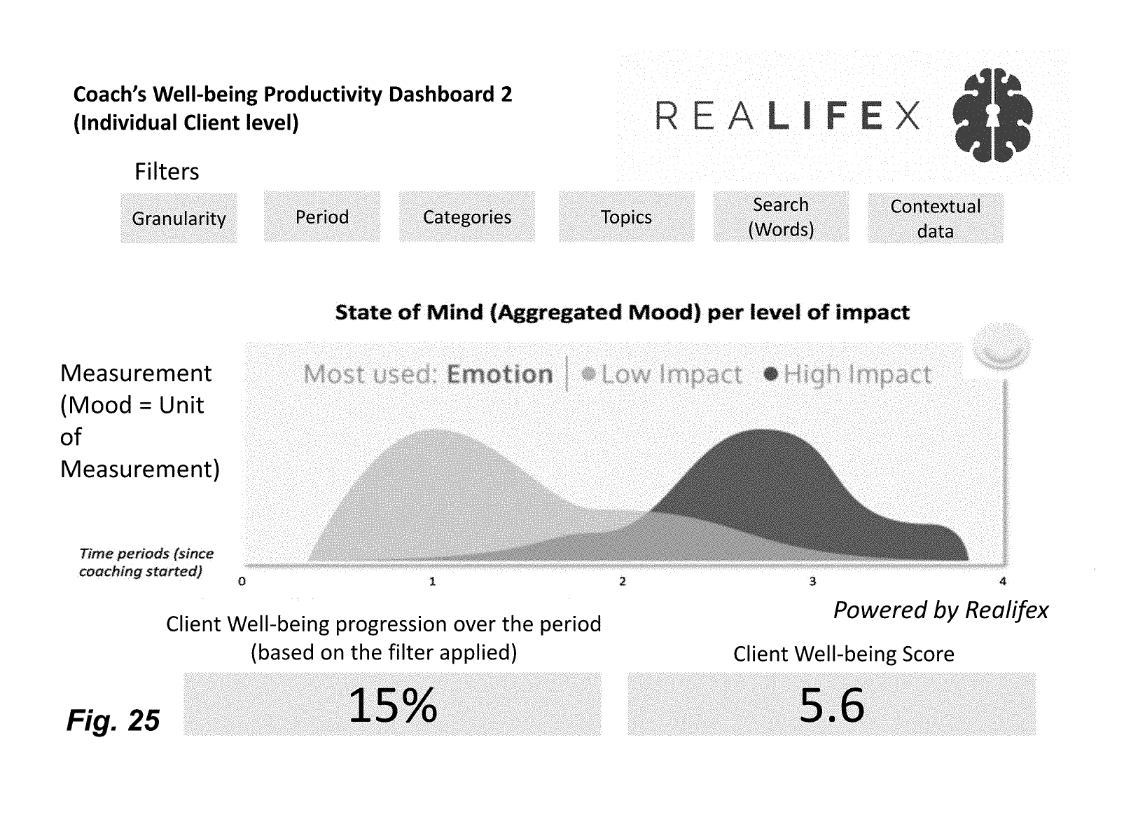

FIG. 25 illustrates a “State of Mind” per level of Impact display interface for individual client.

The instructor user interface (trade name “Real Life Portal”) offers number of advantages in this embodiment:

Real-time deep interactions with a client and a coach

Individual wellbeing coaching (for each client)

Coaching efficiency measurement (across the client base).

As opposed to a classic messaging/communication tool, RealifePortal securely discloses personal notes from clients' life (subject to clients opt-in) to a coach/mentor. The coach observes and analyses their clients' life and can interact whenever necessary to help their clients manage a difficult situation or encourage them when they achieve a goal or improve

The client expresses themselves honestly, for themselves first but knowing that someone is there to support them and to give them confidence. The coach positions themselves beside their clients as a caring presence. Once the client is on track, they can continue expressing themselves in RealifeChange.

Therefore:
The client doesn't write to their coach directly using a real time communication tool. They express themselves, for themselves, but with a caring presence close to them.
The Coach keeps in touch with the reality of their clients' life between meetings and are able to interact/react in context to deliver high quality/relevant coaching based on real life experience.

Individual Well-Being Coaching.

RealifePortal allows a coach to visualise their clients' progress towards their own well-being, through well-being scores and the coach is able to determine what coaching is beneficial for each of their clients and to take action to guide, encourage, adjust and suggest when required to drive positive results towards their own definition of achievement and well-being.

The instructor interface process enables a coach to quantify the efficiency and effectiveness of their coaching/mentoring. See, for example, the displays shown in FIGS. 23, 24, and 25.

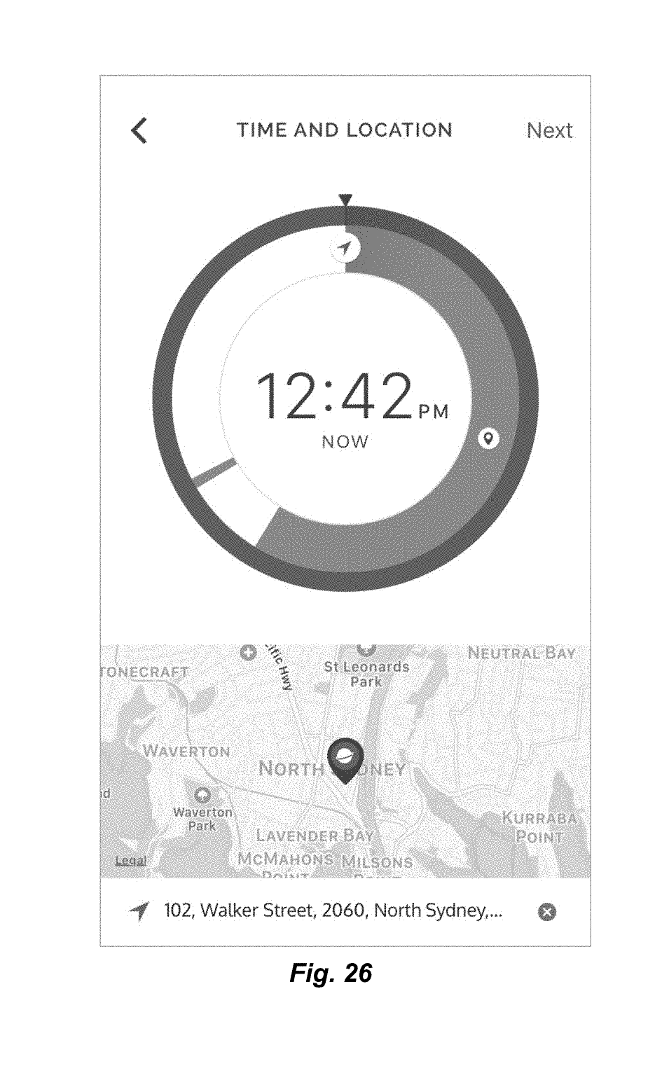

FIGS. 26 through 36 show selections of other interfaces which may be generated by the user interface process for the embodiments discussed above.

FIG. 26 is a Time and a Location dashboard showing current time and current location which the user can actuate for entry of a “Discovery” note in this case.

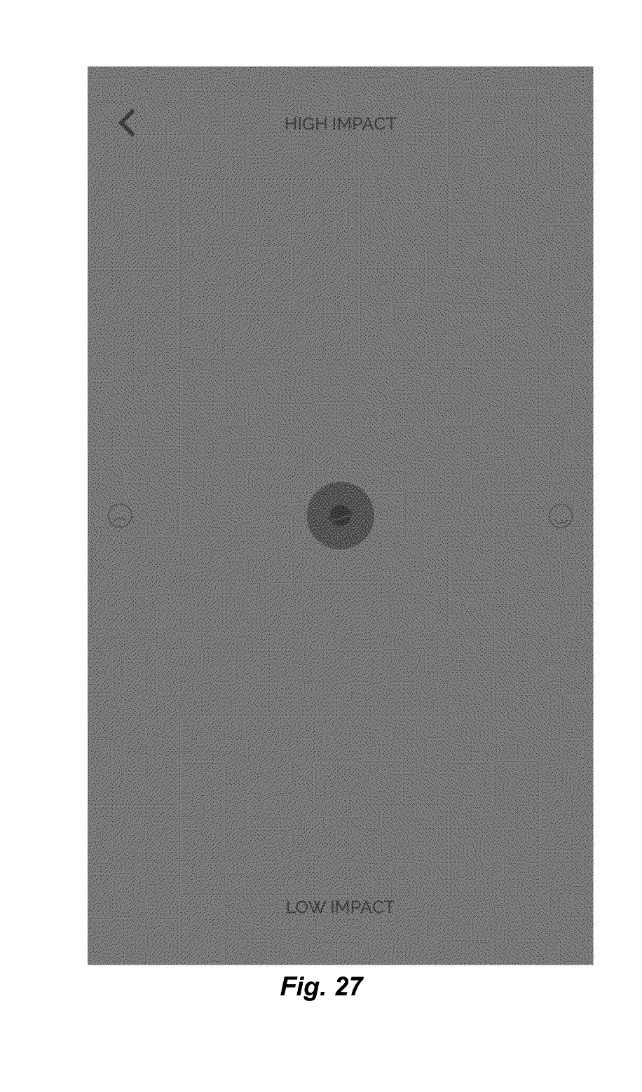

FIG. 27 shows a Discovery Impact and Mood matrix.

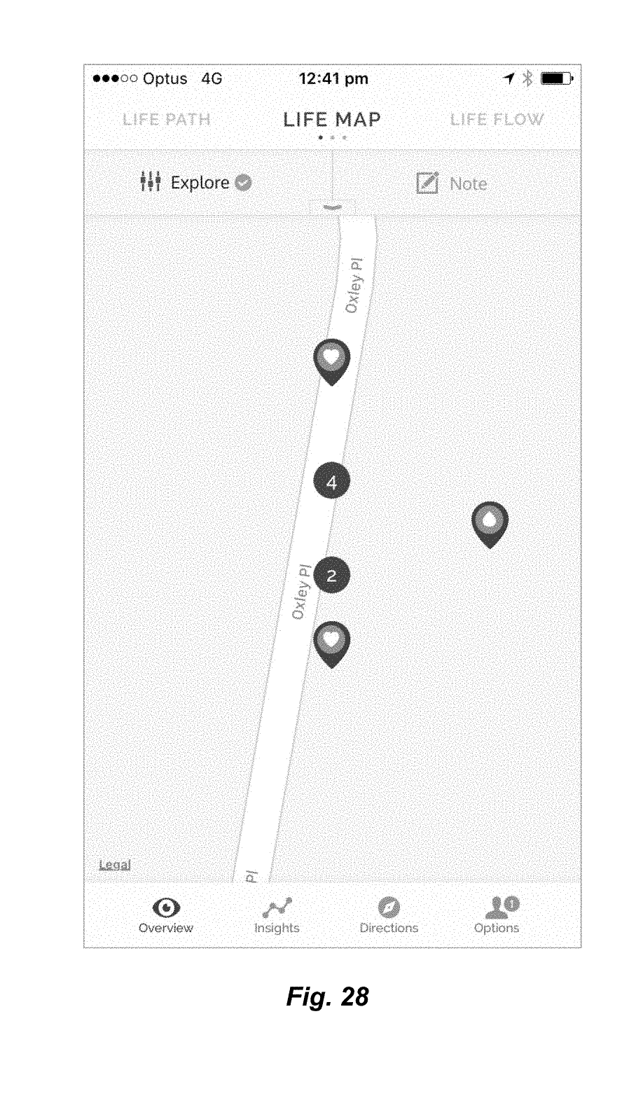

FIG. 28 shows a Life Map interface.

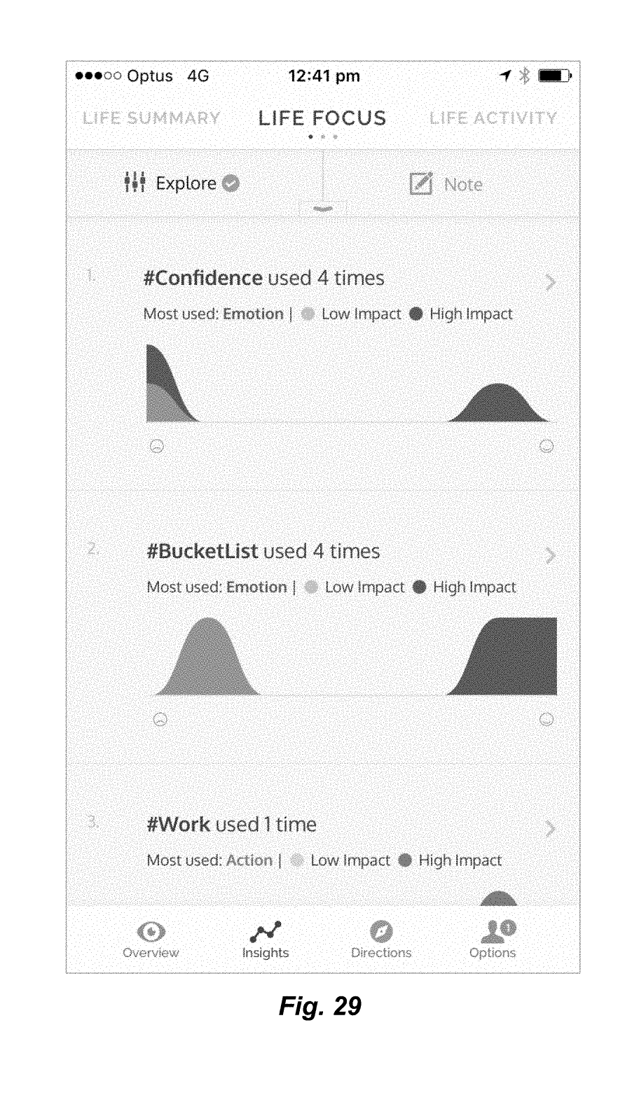

FIG. 29 shows a Life Focus interface.

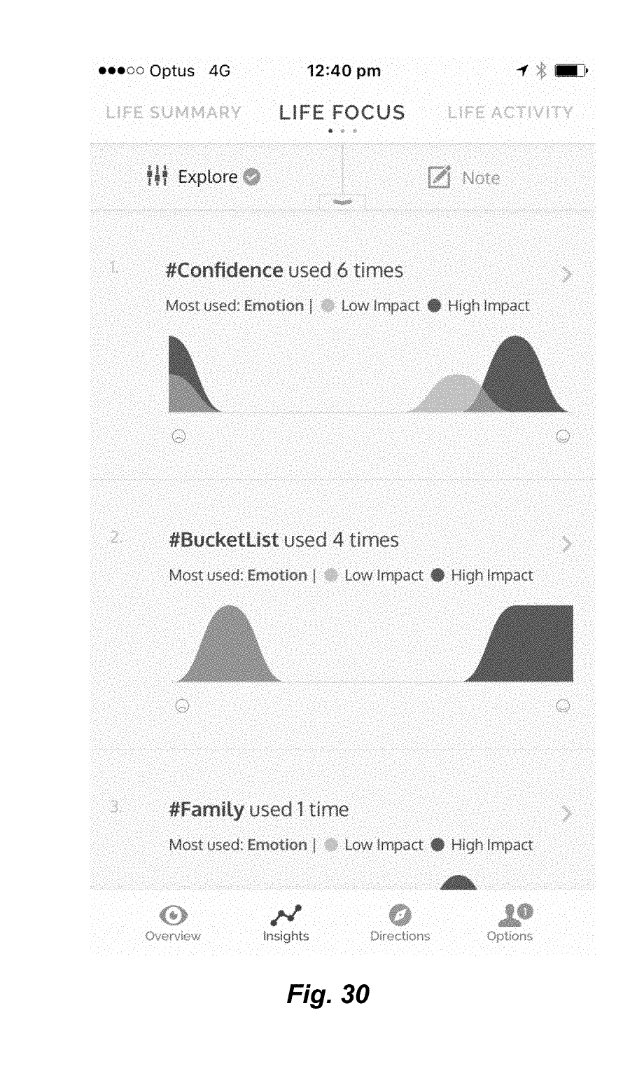

FIG. 30 shows another example of a Life Focus interface.

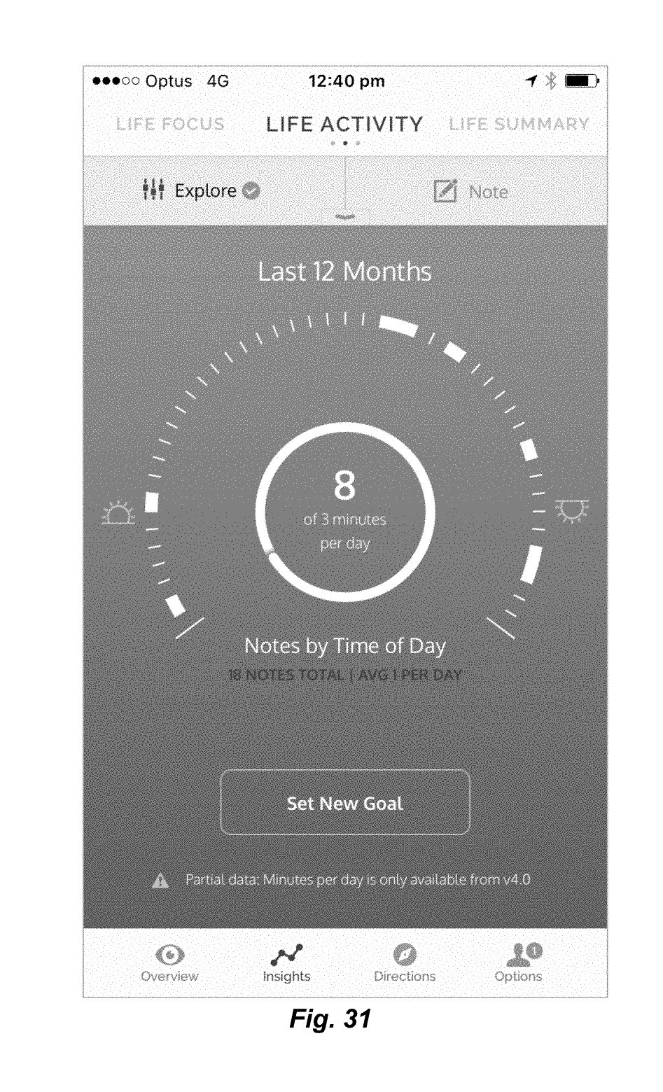

FIG. 31 shows a Life Activity interface, which shows when notes were input by time of day.

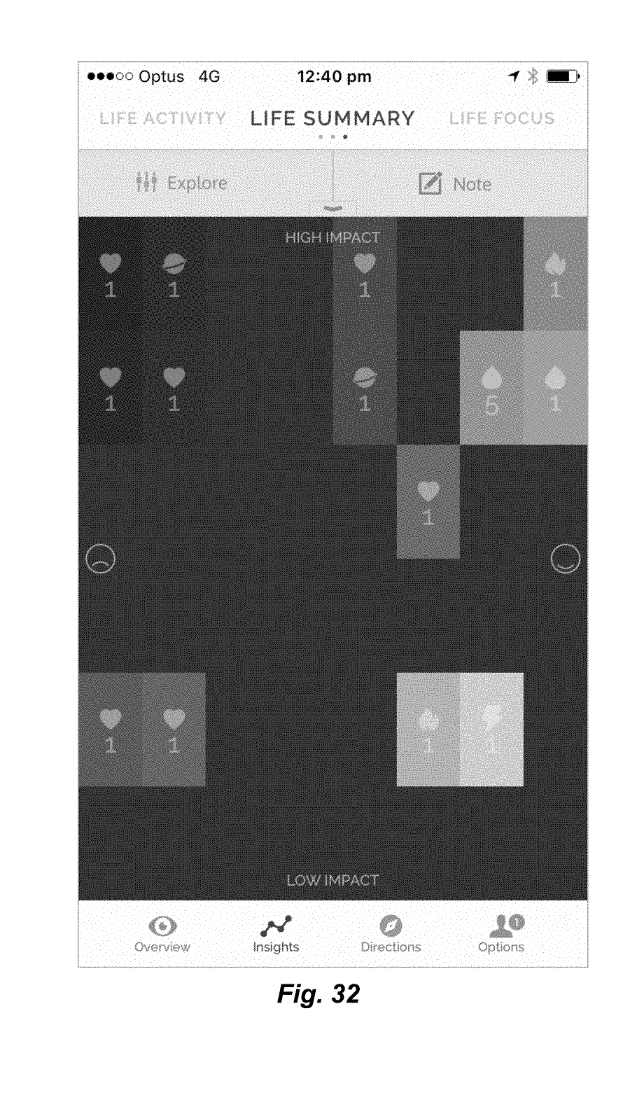

FIG. 32 shows a Life Summary interface. This interface is based on the Impact and Mood metrics. The user can touch one of the segments to “drill down” and find out what input, score etc. was associated with the segment.

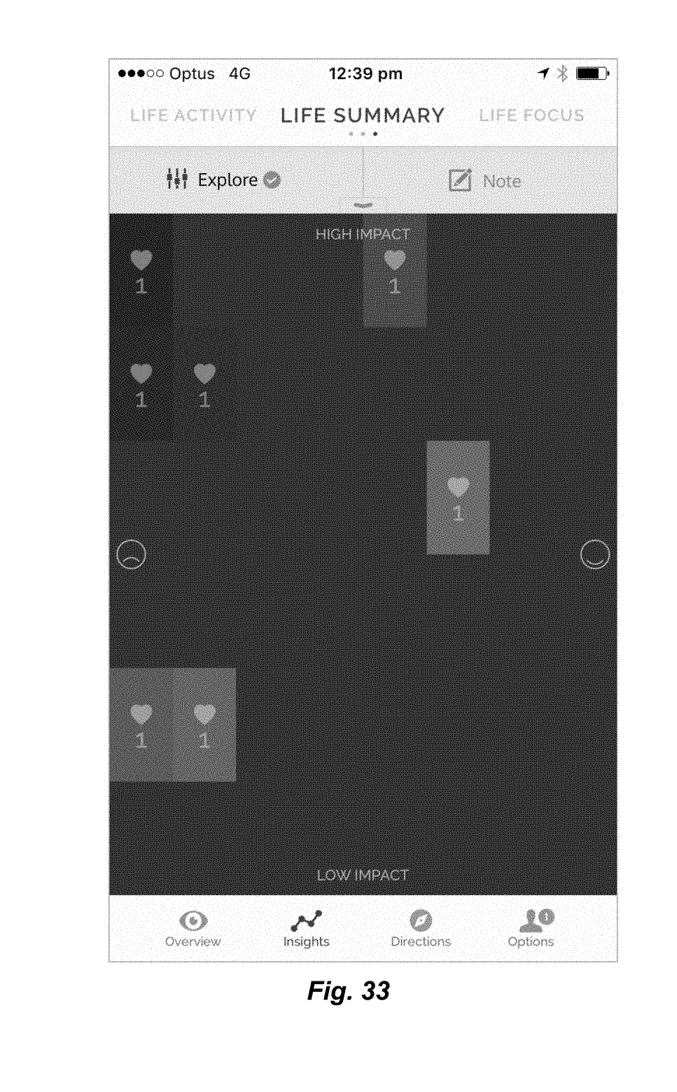

FIG. 33 shows an example of a Life Summary interface for the “Emotion” category.

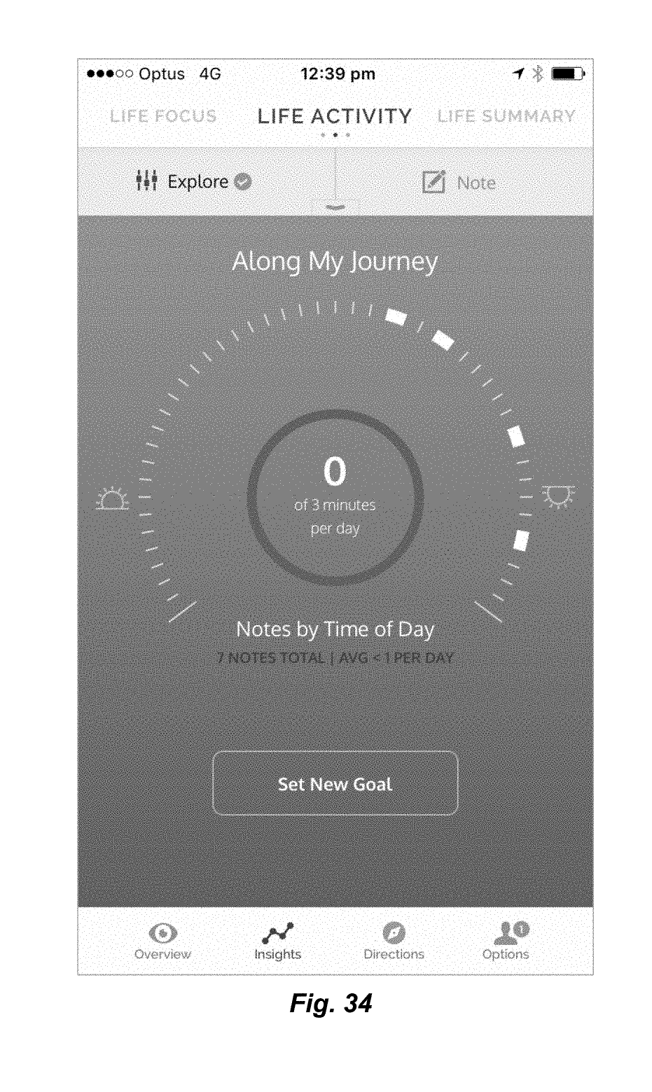

FIG. 34 is another Life Activity interface example.

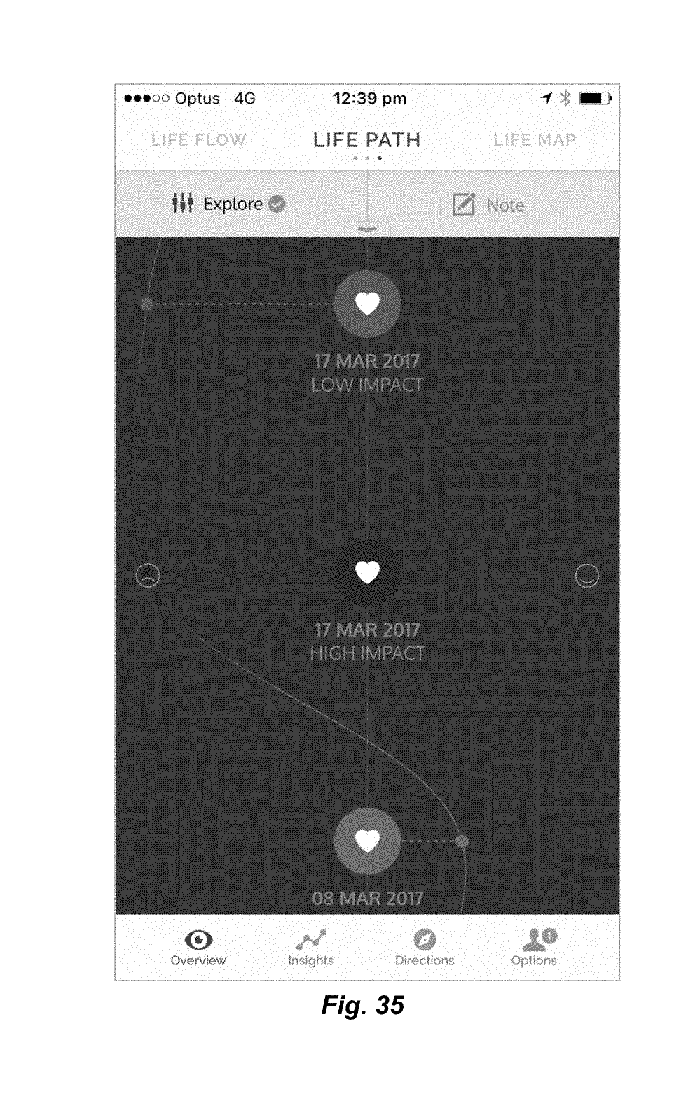

FIG. 35 is another Life Path interface example.

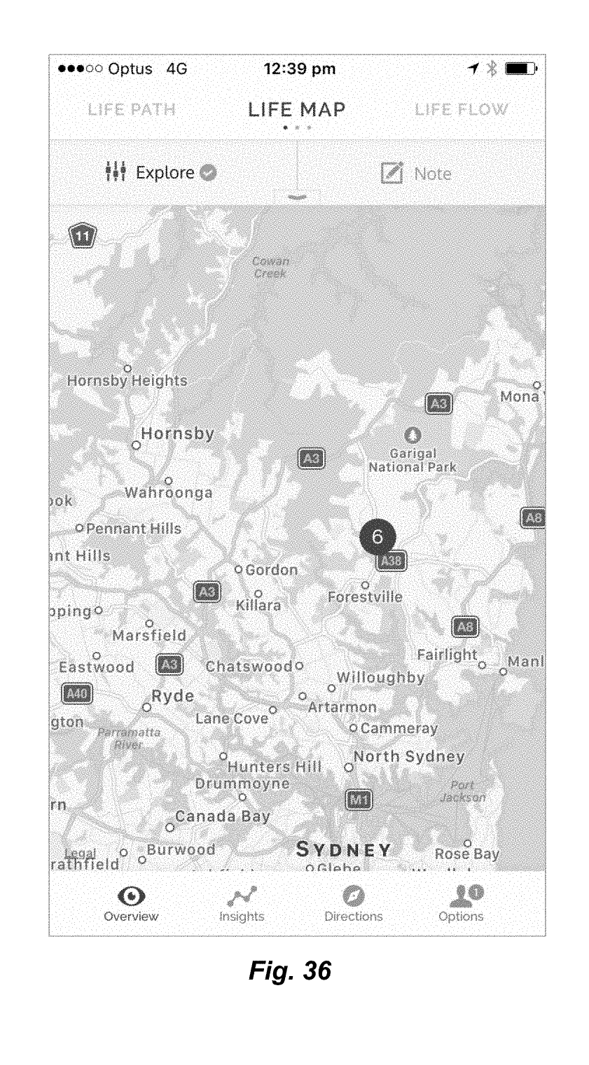

FIG. 36 is a Life Map example.

In an embodiment, the system is also arranged to provide a “program” which comprises a series of suggestions of different types. For example a program is a series of personal suggestions of different types:

Autofocus which Shapes Dashboards Depending on User Focus

Chatbot.

Inside the suggestion, a chatbot will invite the user to ask any question of their life and will answer in real time: How did I feel at work over the last 30 days? What's my most positive/negative topic in life? Which program should I follow next?→based on Life Autofocus rules engine

Emotionally-Driven Suggestions:

depending on the variation (progression and min/max variance) of the well-being suggestions can be sent to the user and/or to the coach/mentor in real-time. Example: If there is a drop in the well-being on the topic family, the suggestion can say: “Ok, I see a negative feeling about your family here, tell ME more about it OR let's go through the issue together (chatbot)→based on Life Autofocus rules engine

Action-Driven Suggestion:

The virtual coach can say: “Create an action now for physical exercise”; “You need to make a decision about this”. A call-to-action will pre-create an action or an emotion note for the user to rate (perception) and comment.

Media Suggestion:

A suggestion in a program can contain a video, soundtrack or text+images

Inside Program Variations:

Depending on the state of mind of the user, the content of the suggestion sent as part of a program will be personalised (merge fields of course but the full suggestion can be different. Example: struggling=>Chatbot; going well=>video

A Program may be suggested to a user depending on their state of mind, defined from notes (for example)—life events, contextual data and previous responses to programs. The system can identify users eligible for a program using a complex rule (e.g. using LifeAutofocus rules engine).

In embodiments, the system may have a number of other innovations:
1. Shared dashboards between coaches/mentors and their clients (via a secure access code that materialise the consent of the client to securely share their life data);
2. 4 filters available to shape well-being score dashboards: Time; Perception/Well-being score; Topics (focus in life) and category (Experience, Action etc. . . . )
3. Several dashboards available which show different dimensions of the Well-being score: overall, by topic, by life impact, by category
4. Smart Graph innovation: Activating the smart graph will make the report more readable (morphing the curve) by consolidating the notes by pre-defined time periods (12 hours, 24 hours, week etc). Disabling smart graph will show all the data points on the graph allowing deeper analysis. Inspired from Fractal science.
5. Clicking on a specific note or group of notes, will show a conversation view with only these selected notes. User experience.

A number of trade made names and trademarks are used in this Specification. They include LifeAutoFocus, RealLifeChange, WellBeingScore, RealLifePortal, LifeMap, LifeFOcus, LifeSummary and others. It will be appreciated that these are trade names only and are not limiting in any way. Embodiments of the invention may be complemented which need to utilise these trade names

In the above embodiment, the application of the system relates to “life coaching”. The invention is not limited to this application. Embodiments of systems in accordance with this invention may be utilized for other domains, including, but not limited to, the following:
Psychologists, psychiatrists
Health care, medical facilities
Insurance and corporate insurance
Medical research laboratiories
Business-employees' wellbeing
Education, high schools
Anti-addiction centers
and more applications.

A number of different dimension data and context data are discussed in the above description. The invention is not limited to these dimensions and context, and any others may be others may be utilised.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A system for monitoring personal activity, comprising a computer processor, a memory and an operating system arranged to support computer processes, a user data input process arranged to receive user input activity data, and a contextual data process, arranged to receive contextual data generated by a contextual data generating device, and to associate the contextual data with the user activity data.

2. A system in accordance with claim 1, comprising an analysis and suggestion process, arranged to analyse the user input data together with the contextual data and determine suggestions based on the user input data and the contextual data.

3. A system in accordance with claim 2, wherein the suggestion process is arranged to present suggestions to the user via a user interface.

4. A system in accordance with claim 2, wherein the analysis process and suggestion process are arranged to analyse the user input activity data and control a user interface process to generate a user interface in dependence on the analysis of the user input activity data.

5. A system in accordance with claim 4, wherein the user interface process is arranged to generate user interfaces in the form of a plurality of dashboards, and the analysis process and suggestion process are arranged to select and generate a dashboard in dependence on the analysis of the user input activity data.

6. A system in accordance with claim 4, wherein the analysis process and suggestion process are further arranged to analyse the contextual data associated with the user input activity data, and control the user interface process, to generate user interfaces in dependence on the analysis.

7. A system in accordance with claim 1, further comprising a dimension data process arranged to generate dimension data and associate the dimension data with the user input activity data, the dimension data comprising a plurality of dimension data categories.

8. A system in accordance with claim 7, when read on to any one of claim 4, or 5, or 6, the analysis process and suggestion process are arranged to analyse dimension data associated with the user input activity data, and control the user interface process to generate user interfaces in dependence on the analysis.

9. A system in accordance with claim 1, further comprising an instructor data input process arranged to receive instructor data for control of user interfaces for presentation to the user, whereby instructor data may be presented to a user.

10. A system in accordance with claim 9, further comprising an instructor data interface process arranged to generate instructor interfaces for presentation to the instructor, and enabling an instructor to receive user input activity data.

11. A non-volatile computer readable medium, providing s computer program, comprising instructions for implementing a system in accordance with claim 1.

12. A system for monitoring personal activity, comprising a computer processor, a memory and an operating system arranged to support computer processes, a user data input process arranged to receive user input activity data, a user interface process arranged to generate user interfaces for presentation to a user, and an analysis process and suggestion process, arranged to analyse the user input activity data and control the user interface process to generate user interface in dependence on the analysis of the user input activity data.

13. A system in accordance with claim 12, wherein the user interface process is arranged to generate user interfaces in the form of a plurality of dashboards, and the analysis process and suggestion process are arranged to select and generate a dashboard in dependence on the analysis of the user input activity data.

14. A system for monitoring personal activity, comprising a computer processor, a memory and an operating system arranged to support computer processes, a user data input process arranged to receive user input activity data, a user interface process arranged to generate user interfaces for presentation to a user, and an instructor data input process arranged to receive instructor data for control of user interfaces for presentation to the user, whereby instructor data may be presented to a user.

15. A system in accordance with claim 14, further comprising an instructor data interface process arranged to generate instructor interfaces for presentation to the instructor, and enabling an instructor to query user input activity data.

16. A non-volatile computer readable medium, providing a computer program, comprising instructions for controlling a computer to implement a system in accordance with claim 12.

17. A non-volatile computer readable medium, providing a computer program, comprising instructions for controlling a computer to implement a system in accordance with claim 13.

18. A non-volatile computer readable medium, providing a computer program, comprising instructions for controlling a computer to implement a system in accordance with claim 14.
